(19) United States
(12) Patent Application Publication
(10) Pub. No.: US 2013/0202639 A1
(43) Pub. Date: Aug. 8, 2013

(54) Synthetic Herpes Simplex Viruses for Treatment of Cancers

(71) Applicants: Konstantin G. Kousoulas, Baton Rouge, LA (US); Jason D. Walker, Baton Rouge, LA (US)

(72) Inventors: Konstantin G. Kousoulas, Baton Rouge, LA (US); Jason D. Walker, Baton Rouge, LA (US)

(21) Appl. No.: 13/634,611

(22) PCT Filed: Mar. 25, 2011
(86) PCT No.: PCT/US11/29939

§ 371 Date: Dec. 18, 2012

Related U.S. Application Data
(60) Provisional application No. 61/317,345, filed on Mar. 25, 2010.

(51) Int. Cl. C12N 7/00; A61K 45/06; A61K 35/76

(52) U.S. Cl. CPC C12N 7/00; A61K 35/763; A61K 45/06
USPC 424/231.1; 435/235.1; 424/93.2

(57) ABSTRACT

New recombinant oncolytic viral vectors have been constructed based on a known herpes simplex virus-1 with a single 34.5 gene and a synctial mutation (called OncSyn (OS) virus), which was designed to be more immunogenic than the parental OS virus largely due to deletion of the viral gene viral host shutoff (vhs) gene (the “OSV” virus). In another embodiment, the OSV virus was constructed to constitutively express 15-PGDH (the “OSVP” virus), the principal enzyme responsible for degradation of PGE2. OSVP was shown to decrease both breast tumors and prostate cancer tumors in mice models. In addition, OSVP was shown to trigger substantial inflammatory cytokine production and pro mote anti-tumor immune responsiveness. These altered viruses, OSV and OSVP, can be used to treat various cancers including breast, prostate, liver, colon, and other tissues. Other exogenous genes can be added to either OSV or OSVP to improve the therapeutic response.

The benefit of the filing date of provisional U.S. application Ser. No. 61/317,345, filed 25 Mar. 2010, is claimed under 35 U.S.C. §119(e) in the United States, and is claimed under applicable treaties and conventions in all countries.

This invention was made with government support under grant number RO1 AI43000 awarded by the National Institutes of Health. The government has certain rights in the invention.

### TECHNICAL FIELD

This invention pertains to new, safer oncolytic herpes simplex viruses that enhance immune responses against viral infected cells and tumor cells, and that can carry exogenous genes for gene therapy, to increase the immune response, or to present antigens for a vaccine.

### BACKGROUND ART

An oncolytic virus is a virus that preferentially infects and lyses cancer cells. The virus can be effective against tumors both by direct destruction of cancer cells, and if a vector, by enabling genes that express proteins that are delivered to the tumor site. An oncolytic virus is usually modified to have selective replication only in tumor cells. For example, oncolytic human herpes simplex viruses have been genetically engineered to limit neurovirulence, establishment of latency and reactivation, and to replicate exclusively in cells with deficient apoptotic mechanisms (i.e., cancer cells) (5, 6, 37, 78, 79, 80, 81, 85). These genetic changes are fundamental to the safety and efficacy of oncolytic herpes viruses, but often result in rapid clearance of the virus by the host immune response, thus limiting its therapeutic potential. However, it is precisely the antiviral immune response that is thought to help overcome tumor-induced immune suppression allowing for anti-tumor immunity to develop. In this regard, HSV-1 infections within tumors function as in situ vaccines providing the necessary inflammatory signals that engage innate and adaptive immune responses to tumor antigens. Thus, oncolytic HSV-1 can be effective both directly as a cancer killing agent, and indirectly as an immunological enhancer, or in situ cancer vaccine (13, 49, 77, 84). Previously, the fusogenic oncolytic herpes virus OncSyn (OS) was shown to be effective at treating primary solid breast tumors in mice (25, 26; see also, International Application Publication No. WO 2008/14151). Moreover, treatment of primary 4T1 tumors in syngeneic Balb/c mice with OS lead to substantial reduction in the formation of metastatic foci within multiple organs and in some cases eliminated lung metastasis, suggesting the development of effective anti-tumor immunity (43).

The HSV-1 vhs (viral host shut-off) gene encoded by the UL41 open reading frame has multiple functions that are known to suppress anti-viral immune responses: (1) vhs protein is an RNase that degrades viral and cellular mRNAs and thus limits host and viral antigen production (3, 32, 33, 45, 48, 51, 56, 59, 61, 65, 75); (2) UL41(vhs) with ICP47 is known to inhibit MHC-I antigen presentation (59), while vhs alone has also been implicated in reduction of MHC-II expression (69); and (3) vhs has been reported to suppress production of cytokines and chemokines and inactivate dendritic cells (7, 59). In agreement with these reports, deletion of the vhs gene prevented HSV-1 mediated inactivation of antigen presentation by dendritic cells (DC) (54) and improved the immunogenicity of a candidate replication-defective HSV-1 vaccine strain (16, 55). Furthermore, deletion of the vhs gene causes substantial reduction in neurovirulence (48, 60, 64).

It is generally assumed that advanced tumors promote the formation of an immunosuppressive and tolerogenic microenvironment that subverts the innate immune response leading to inhibition of anti-tumor adaptive immune responses (14, 46, 76). Tumor-derived immunomodulation may include (1) alteration of tumor antigen expression to render tumor cells less detectable by the immune system, (2) secretion of factors and cytokines that inhibit dendritic and T-cell functions, and (3) induction of immune cells that can suppress anti-tumor immune responses including MDSC and regulatory T cells (Treg) (44). Viral vectors have been designed to overcome this suppression of the immune system (37).

A prominent immunosuppressive factor in many cancers is prostaglandin 2 (PGE2). PGE2 is a short-lived lipid based signaling molecule with potent localized paracrine and autocrine functions that is particularly important for tumor development. PGE2 has been shown to promote tumor angiogenesis (38, 71), drug resistance (36), invasion and migration (35), immune suppression (24, 72, 73), and the inhibition of apoptosis (34). PGE2 is generated by tumors and tumor-associated immune cells from arachidonic acid with the rate-limiting step being the enzymatic activity of cyclo-oxygenase 2 (COX-2). PGE2 is negatively regulated by rapid conversion to 15-keto metabolites by 15-prostaglandin dehydrogenase (PGDH), an enzyme with both intra- and extra-cellular functions. 15-PGDH is a tumor suppressor (2, 9, 42, 74), and loss of 15-PGDH expression in a variety of cancers often accompanies COX-2 upregulation and is correlated with disease progression (2, 35, 66, 67, 70). Elevated levels of tumor-associated COX-2, coupled with a loss of PGDH expression allows many tumors to maintain high levels of PGE2, which is a poor prognostic indicator and is considered to be an important step in the evolution of malignant cancers (reviewed in (21)). COX-2 activity in tumors may be blocked using selective or non-selective inhibitors whose use has been shown to significantly decrease cancer risk for a wide range of cancer types (reviewed in (22, 23) respectively); however systemic COX-2 inhibition has side effects that are poorly tolerated. Transient localized restoration of PGDH expression in tumors using targeted adenoviral vectors has been shown to inhibit PGE2 accumulation, tumor angiogenesis and growth (27). Overall, tumor-associated PGE2 is strongly associated with immune suppression and cancer progression. Novel anti-cancer immune therapies designed to limit PGE2 signaling can promote stronger immune responses and inhibit tumor development. In a mouse model system for colorectal tumors, mice treated with an adenovirus expressing murine 15-PGDH developed anti-tumor immune responses that caused eradication and long term survival in 70% of the mice (10).

Breast cancer is the most common cancer among women, excluding cancers of the skin, accounting for nearly 1 in 3 cancers diagnosed in US women. In western countries breast cancer is the second leading cause of cancer death in women and is associated with high morbidity and mortality. A new and promising strategy for cancer therapy is the use of modified viruses that have been engineered to selectively replicate within cancer cells (oncolytic virotherapy). A number of viruses have been explored as tumor-selective replicating vectors, including adenovirus, herpes simplex virus type-1 (HSV-1), vaccinia virus, reovirus, Newcastle disease virus, vesicular stomatitis virus, measles virus, poliovirus and West Nile virus. Multiple murine tumor models have been used as preclinical settings for therapeutic purposes. The 4T1 mammary carcinoma model has several distinct advantages to be used as such model. It is regarded as a highly physiological, clinically-relevant mouse model that closely resembles stage 1V human breast cancer in its properties (1). 4T1 cells are considered to be very weakly immunogenic (relative antigenic strength is less than 0.01 with 9.9 being the most immunogenic), and they spontaneously metastasize to distal parts of the body (1, 50).

Several cancers have been treated with modified herpes simplex virus. For example, brain, breast, prostate, colorectal, glioblastoma, head and neck cancers. (77, 83, 84) In addition, taxanes and other chemotherapeutics have been shown to be synergistic with some oncolytic virus treatments. Pretreatment with paclitaxel was shown to lead to the formation of gaps between neighboring tumor cells, which allowed for better penetration of oncolytic herpes virus into the tumor (86). Additionally, when docetaxel was used in combination with an oncolytic herpes virus, G474, the dose of G474 was reduced by a factor >10 without limiting efficacy in treating mouse tumors (87). Similar results have also been reported for paclitaxel (88).

U.S. Pat. Nos. 5,328,688 and 6,120,773 disclose a recombinant herpes simplex virus vaccine based on the making the virus avirulent by prevention of expression of the γ₁ 34.5 gene.

U.S. Pat. No. 5,585,096 discloses a mutant herpes simplex virus with a defective expression of the γ₁ 34.5 gene and the ribonucleotide reductase gene.

International Publication No. WO 98/04726 discloses a herpes simplex virus strain that is disabled by inactivating both ICP34.5 and ICP27 genes for use as a gene delivery vector.

U.S. Patent Application Publication No. 2002/0019362 discloses treatment of cancers with a herpes simplex virus that has an alteration in the γ₁ 34.5 gene.

U.S. Pat. No. 6,846,670 discloses a genetically engineered herpes virus vector for treatment of cardiovascular disease that is modified by lacking a γ₁ 34.5 gene and operably comprises a heterologous nucleic acid.

U.S. Patent Application Publication No. 2006/0188480 discloses a herpes virus that lacks a functional ICP34.5 gene and is capable of delivering two genes which, in combination, enhance the therapeutic effect. The two genes are chosen from a gene which encodes a prod-drug activating enzyme, a gene which encloses a protein capable of fusing cells, and/or a gene which encodes an immunomodulatory protein.

U.S. Patent Application Publication No. 2007/0031383 discloses a recombinant herpes simplex virus expressing only a single γ₁ 34.5 gene and an expressible cytokine-encoding DNA.

International Application Publication No. WO 2008/141151 discloses the structure and methods of using the OncSyn (OS) mutant virus.

### DISCLOSURE OF INVENTION

We have constructed a new recombinant oncolytic viral vector based on the OncSyn (OS) virus, designed to be more immunogenic than the parental OS virus largely due to deletion of the viral gene viral host shutoff (vhs) gene, and named the virus OSV. In addition, we used OSV to add a gene to constitutively express 15-PGDH, the principal enzyme responsible for degradation of PGE2, and named the virus OSVP. Infection with OSVP of both breast and prostate tumors implanted in mice inhibited tumor growth. OSVP treatment was also shown to decrease metastasis, trigger substantial inflammatory cytokine production, and promote anti-tumor immune responsiveness. These altered viruses, OSV and OSVP, can be used to treat various cancers including breast, prostate, liver, colon, throat, and other tissues. Exogenous genes, other than PGDH, can be added to OSV or to OSVP by insertion into the deleted vhs open reading frame. These exogenous genes can lead to expression of proteins that can, for example, inhibit HSV or cancer suppression of the immune response, present a specific cancer or other antigen to the host immune system, or upregulate the immune response. Expression of these exogenous genes can be under control of the HSV promoters or an exogenous promoter. The exogenous promoter can be chosen to target specific tissues or tumors. The oncolytic viral treatment can be combined with other cancer treatments, e.g., radiation or chemotherapy, to increase the therapeutic response and further decrease the tumor size.

### BRIEF DESCRIPTION OF THE DRAWINGS

FIGS. 1A-1C is a schematic representation of the genomic structure of oncolytic recombinant viruses OS, OSV and OSVP. FIG. 1A, the top line, represents the prototypic arrangement of the HSV-1 OncSyn (OS) genome with the unique long (UL) and unique short (US) regions flanked by the terminal repeat (TR) regions. The A denotes the approximate location of the OS genomic deletion between the UL and US regions and the position of the gB syncytial mutation is indicated. The expanded area below the depicted genome shows the genomic region from 84531-96068 nucleotides encompassing the UL38-UL43 genes. FIG. 1B represents the genomic organization of the OSV recombinant virus showing deletion of the UL41 gene. FIG. 1C represents the genomic organization of the OSVP recombinant virus showing insertion of the 15-PGDH gene cassette, which includes SV40 and CMV promoter, in-place of the deleted UL41 sequences.

FIG. 2A shows the morphology and growth of nearly confluent Vero cell monolayers infected with wild-type HSV-1 (F), OS, OSV, or OSVP viruses at MOI of 0.001, as the individual viral plaques were visualized 48 hr post infection by immunohistochemistry and photographed at the same magnification with a phase contrast microscope.

FIG. 2B shows the viral titers (PFU/ml) for triplicate cultures collected at 48 hr post infection of nearly confluent 4T1 cell monolayers infected with OS, OSV, or OSVP viruses at an MOI of 5.

FIG. 3 illustrates the level of PGE2 (pg/ml) as measured in nearly confluent monolayers of 4T1 cells infected with OS, OSV, or OSVP viruses at a MOI of 5, and harvested and assayed for PGE2 2 days post infection by ELISA immunoassay. “P” denotes transient expression with a 15-PGDH mammalian expression vector, while “No Tx” denotes mock-treated cells. PGE2 assays were performed in triplicates and error bars represent the 95% confidence interval (CI) of the mean.

FIG. 4 illustrates the change in tumor volume, measured using a digital caliper at defined time intervals prior and after treatment (X axis), in Balb/c mice implanted subcutaneously in the interscapular area with 1×10⁵ viable 4T1 cells. When tumors reached approximately 80-90 mm³ in volume, the tumors were injected with each virus (1×10⁷ PFU/ml in PBS buffer) or PBS alone at days 1, 3 and 6. The asterisk indicates statistical significance by non-parametric analysis. The results shown are from one of three independent experiments that produced similar results.

FIG. 5 illustrates the metastatic loads calculated by the total pulmonary clonogenic metastatic foci enumerated by limited dilution culture in the presence of 6-thioguanine from lungs harvested from virus-treated mice bearing 4T1 tumors that were sacrificed 30 days post tumor inoculation. The metastasis incidence rate for each group is indicated below the x-axis. Means for each group are represented by the horizontal bars with error bars delineating 95% CI. The results shown are from one of three independent experiments that produced similar results.

FIGS. 6A-6D illustrates the immune response in 4T1 tumor bearing mice with similar-sized, and well developed tumors measured in cells isolated two days after being given a single intratumoral injection of the control (PBS) or of the OS, OSV, or OSVP viruses. FIGS. 6A-6B illustrate specific immunogenic cells from draining lymph nodes. FIG. 6A shows the CD83 activation marker, and FIG. 6B shows the CD4+CD25+FoxP3+ regulatory T cells. FIGS. 6C and 6D illustrate specific immunogenic markers isolated from splenocytes. FIG. 6C shows the CD83 activation marker, and FIG. 6D shows the MDSC markers (Gr1⁺, CD11b⁺). Indicated phenotypes are shown as the mean percentage of total live cells. Error bars represent 95% CI of the means, and n=3 unless otherwise noted.

FIGS. 7A-7H illustrate the cytokine immunoprofiles of lymphocytes from draining lymph nodes derived from mice with similarly-sized 4T1 tumors (200-300 mm²) that were treated twice with a 3 day interval with OS, OSV, OSVP or PBS (control), and isolated two days after the second treatment. The lymphocytes were isolated and cultured alone at a concentration of 100,000 cells per ml. After culturing for 24 hours, supernatants were assayed for TH1/TH2 cytokine production by Bioplex (Bio-Rad). Shown are the mean quantities of the indicated cytokines in pg/ml with error bars representing 95% CI (n=3).

FIGS. 8A-8H illustrate the cytokine immunoprofiles of lymphocytes from draining lymph nodes derived from mice with similarly-sized 4T1 tumors (200-300 mm²) that were treated twice with a 3 day interval with OS, OSV, OSVP or PBS (control), and isolated two days after the second treatment. The lymphocytes were isolated and cultured with immobilized anti-CD3 stimulatory antibody at a concentration of 100,000 cells per ml. After culturing for 24 hours, supernatants were assayed for TH1/TH2 cytokine production by Bioplex (Bio-Rad). Shown are the mean quantities of the indicated cytokines in pg/ml with error bars representing 95% CI (n=3).

FIG. 9 illustrates the change in tumor volume over time (days), measured using a digital caliper at defined time intervals prior and after treatment (X axis), in male syngeneic C57BL/6 mice implanted subcutaneously on the dorsum with 0.5×10⁶ viable RM-9 prostate cancer cells. When tumors reached approximately 80-90 mm³ in volume, the tumors were injected with either control or the OSVP virus (1×10⁶ PFU/ml in PBS buffer) at days 2, 6, and 10. Shown are the mean tumor sizes with error bars representing 95% CI.

### MODES FOR CARRYING OUT THE INVENTION

As described in WO 2008/141151, we previously developed the HSV-1 recombinant virus, HSV-1OncSyn (OncSyn or OS). The OncSyn virus has one of the two γ₁ 34.5 genes as well as adjacent sequences deleted, and carries a syncytial mutation within the UL27 gene encoding gB. In addition, one of the two genomic regions coding for the latency associated transcripts (LAT) was deleted. The OncSyn virus was shown to replicate efficiently in human breast cancer cells in cell culture yielding higher viral titers than either the wild-type HSV-1 (F) or parental Onc viruses. Viral glycoproteins including gB were efficiently expressed on cell surfaces indicating that the remaining single γ₁ 34.5 gene adequately supported intracellular glycoprotein transport and cell-surface expression. Importantly, the OncSyn virus spread substantially better in breast cancer cells than in Vero cells producing large syncytial plaques. Intra-tumor injections of the OncSyn virus within xenografts of human breast cancer cells injected into nude mice showed reduction of tumor size, and extensive necrosis of tumor cells. The results showed that the constructed OncSyn virus can effectively kill tumor cells both in vitro and in vivo. Intratumoral delivery of the OncSyn virus produced a significant therapeutic effect as evidenced by the drastic reduction of treated tumors. The OncSyn virus was able to infect, replicate, and effectively fuse and destroy the human breast cancer cells in vitro and in vivo. The OncSyn virus was also re-isolated as a bacterial artificial chromosome (pOncSyn), which enabled the rapid construction of additional viruses. (25, 26, and WO 2008/141151).

As explained in WO 2008/141151, the OncSyn virus has some similarities, as well as substantial differences in comparison to another attenuated HSV-1 virus, NV 1020. (8) Both OncSyn and R7020 viruses are derived from the HSV-1 (F) strain, with the exception that OncSyn virus was derived from the HSV-1 (F) genome cloned into a bacterial artificial chromosome (pYEbac102). Extensive restriction analysis and DNA sequencing of the pYEbac102-derived virus did not reveal any significant genomic changes, suggesting that either the insertion of the bac (bacterial artificial chromosome) backbone or other nucleotide changes may potentially contribute to the observed attenuation characteristics (unpublished observations). Both NV1020 and OncSyn contain a large deletion of approximately 16-kilobase-pairs (Kbp) across the joint region of the long-L and short-S components of the viral genome. This deleted region includes the UL56 gene, and one of the two copies of a 0, γ₁ 34.5 and a 4 genes. In addition, this deletion also includes the entire genomic region coding for one of the two loci encoding the latency-associated transcripts (LAT). The NV1020 virus contains within the deleted genomic region a 5.2 Kbp DNA fragment of HSV-2 and an exogenous copy of the HSV-1 viral thymidine kinase (TK) under the control of the α4 promoter (the native TK gene has been deleted). In contrast, the OncSyn virus contains within the deleted genomic region an insertion of a gene cassette coding for the red fluorescence protein under the constitutive control of the EF-1α promoter, while the native TK gene remains unaltered. The presence of both HSV-1 and HSV-2 glycoproteins gD, gI and gE in the NV 1020 virus may lower the relative efficiencies of intracellular virus assembly and egress, and result in virus attenuation and decreased intra-tumor spread. Alternatively, it is possible that the presence of the HSV-2 viral glycoproteins, especially gD and gE/gI, may broaden the host-range of the recombinant virus. Furthermore, it is believed that the OncSyn virus may be delivered in lower viral doses than the NV 1020 virus due to its potential advantage in virus production and intratumor spread over the NV 1020 virus.

We have now developed new oncolytic viruses to enhance the immune response against viral infected cells and potentially tumor cells. One new virus, called OSV, is based on the OncSyn (OS) virus, but rendered safer for human use by deleting the single copy of the Viral Host Shutoff (VHS) gene (UL41). This deletion attenuates the virus, but also prevents the virus from shutting off host protein synthesis which allows better antigen expression for recognition by the immune system. The vhs-deleted OSV genome contains an approximately 11 kbp deletion encompassing the UL-US junction and the UL41(vhs) deletion (2.5 kbp) for a total of approximately 13.5 kbp deleted sequences. Therefore, this virus is amenable to insertion of multiple gene cassettes expressing genes of interest to cancer therapy without exceeding viral DNA packaging limits. Using the OSV virus as shown in the examples below, we added exogenous genes to the viral genome inserted in the VHS open reading frame (ORF) site. Exogenous genes could also be added at other sites in the viral genome. The addition of the PGDH gene and promoters to the OSV virus was used to upregulate the immune response against both the tumor infected cells and tumor antigens. An increase in PGDH levels decreases the available PGE2 and lowers the immune suppression that PGE2 would otherwise cause.

We successfully replaced the VHS ORF of OSV with DNA sequences encoding murine 15-PGDH. In vitro experiments described below in the Examples show that OSVP nearly eliminated PGE2 accumulation in cultured tumor cell supernatants. In vivo experiments using an immune-competent murine tumor model of breast cancer and prostate cancer as described below showed that the OSVP virus effectively inhibited tumor growth.

Using the OSV virus, one or more exogenous genes can be added to the viral genome in the VHS ORF for expression in the host. Multiple genes can be inserted whose total nucleic acids are preferably from about 10 kbp to about 20 kbp. The preferred addition would be no more than about 5 exogenous genes. The exogenous gene added to the OSV virus can be a gene whose expression affects the function of the host immune system. For example, the expressed protein can decrease the viral suppression of the host immune system, e.g., the addition of a PGDH gene to lower the concentration of viral-induced PGE2. The exogenous gene can also encode for a known immunogenic protein, e.g., interleukin-2 (IL-2), interleukin-4 (IL-4), interleukin-10 (IL-10), interleukin-12 (IL-12), granulocytes-macrophage colony stimulator factor (GM-CSF), Interferon-γ (INF-γ), tumor necrosis factor alpha (TNFα), and co-stimulating surface antigen (CD80) (37). The exogenous gene can also encode for a known anti-angiogenic factors since solid tumor growth depends on the growth of blood vessels, e.g., endostatin, trichostatin A (TSA), vasculostatin (Vstat120), brain-specific angiogenesis inhibitor 1 (BAI1). (37) Finally, the exogenous gene can also encode for a known cancer or pathogen-related antigen which would effectively make the viral vector a vaccine (e.g., HER2/neu (ErbB-2), carcinoembryonic antigen (CEA1), MUC-1, epithelial tumor antigen (ETA), Epstein Barr viral antigens, Papilloma viral antigens, additional herpes simplex viral antigens, and other known viral antigens). One or more of the above exogenous genes can be added to OSV.

The exogenous genes added to OSV can also contain a promoter sequence to enhance the exogenous gene expression. It is believed that a promoter is not necessary for expression since the exogenous gene would fall under the influence of the surrounding HSV-1 genome and would be expressed similarly to the vhs gene during infection. The added promoter sequence can be a ubiquitous promoter, e.g., cytomegalovirus immediate early promoter (CMV-IE), simian virus 40 promoter (SV40), the Rous sarcoma virus long terminal repeat promoter (RSV-LTR), Moloney murine leukemia virus (MoMLV) LTR, other retroviral LTR promoters, phosphoglycerate kinase (PGK) promoter, and other eukaryotic promoters such as the Elongation factor 1a (ER1a). (82) Alternatively, the promoter can be a tissue specific promoter or a tumor specific promoter (e.g., Her-2/neu (erbB2) promoter, carcinoembryonic antigen (CEA) promoter; PSA promoter; probasin (ARR2PB) promoter). Many such promoters are known in the art. (82).

The treatment of cancers with the above HSV virus based on OSV or OSVP can be combined with traditional cancer treatments such as radiotherapy and chemotherapy. For example, known cancer drugs can be administered during the viral treatment, e.g., taxane agents (e.g., docetaxel and paclitaxel), fludarabine, CD20 antibody, histone deacetylase inhibitors, doxorubicin, cisplatin, Trichostatin A (TSA), bevacizumab (Avastin) and other anti-angiogenic drugs. In addition, simultaneous treatment with a NSAID (non-steroidal anti-inflammatory drug) can be beneficial, e.g., treatment with aspirin, ibuprofen, celecoxib, rofecoxib, salsalate, sodium salicylate, and other NSAIDS.

Examples of uses of the new viruses for treatment include treating non-human animals and humans suffering from tumors (neoplasms). Preferentially the virus would be administered by subcutaneous injection, by direct intratumoral injection, or by intravascular injection proximal to the tumor. A typical composition for such injection would comprise the virus and a pharmaceutically acceptable vehicle, which can either include aqueous and non-aqueous solvents. Aqueous vehicles include water, saline solutions, sugar solutions (e.g., dextrose), and other non-toxic salts, preservatives, buffers and the like. Non-aqueous solvents include propylene glycol, polyethylene glycol, vegetable oils, and other non-toxic organic solutions.

The amount of virus to be administered, both in terms of the number of treatments and the dose for each treatment, will depend on the size and health of the subject and the type and location of the tumor. Oncolytic viral therapy has been used for many mammalian solid tumors, for example, breast tumors, pancreatic tumors, prostate tumors, brain tumors, peritoneal tumors, and colorectal tumors. (82)

The invention is also directed to methods of treating neoplastic diseases or tumors by administering the recombinant herpes simplex viruses as described above to patients with such diseases or tumors.

### Example 1

### The OncSyn (OS) Virus

### Synthesis

Cells, viruses and plasmids. African green monkey kidney (Vero) cells, human breast cancer cells (Hs578T), and mouse mammary tumor cells (4T1) (1) were obtained from the American Type Culture Collection (Manassas, Va.). The human breast adenocarcinoma line MDA-MB-435-luc expressing luciferase (MM4L) was kindly provided by Dr. C. Leuschner (Pennington Biomedical Research Center, Baton Rouge, La.). Vero and Hs578T cells were maintained in Dulbecco's modified Eagle's medium (Gibco-BRL; Grand Island, N.Y.), supplemented with 10% fetal bovine serum (FBS) and antibiotics. 4T1 cells were maintained in RPMI 1640 medium (Hyclone, Logan, Utah) containing 10% FBS. The cultures were maintained at 37° C. in a humidified atmosphere of 5% CO₂/95% air. MM4L cells were cultured with Leibovitz's L-15 medium (Hyclone, Logan, Utah) containing 10% FBS. These cells were cultured in tightly closed flasks in a 37° C. incubator. The plasmid pYEbac102 containing the HSV-1 (F) (Tanaka et al., 2003) viral genome was kindly provided by Dr. Y. Kawaguchi (Tokyo Medical and Dental University, Tokyo, Japan). The plasmid pRB3410 was kindly provided by Dr B. Roizman (University of Chicago, Chicago, Ill.). All viruses were routinely grown and titrated in Vero cells. The plasmid encoding red fluorescent protein (pHcRed1-N1) was obtained from BD Biosciences, Clontech (Palo Alto, Calif.). The pEF6/V5-His-TOPO plasmid was obtained from Invitrogen (Carlsbad, Calif.). See also WO 2008/141151.

Construction and Genomic Characterization of Recombinant Viruses HSV-1Onc (Onc) and HSV-1OncSyn (OncSyn).

The red fluorescent protein (RFP) gene was PCR-amplified from plasmid pHcRed1-N1 and cloned into the plasmid pEF6/V5-HisTOPO. Subsequently, the RFP gene under the elongation factor 1-alpha (EF-1α) promoter was cloned into the pRB3410 XbaI site producing plasmid pJM-R. In this plasmid the RFP gene cassette interrupts the viral sequence creating two 1838 and 2300 bp viral DNA flanking segments to facilitate homologous recombination with the viral genome.

Vero cells were transfected with pJM-R and twenty-four hours post-transfection cells were infected with the pYEbac102-derived virus. Red virus plaques formed on Vero cells were collected and sequentially plaque-purified at least six times. The resultant virus was named HSV-1Onc (Onc). An HSV-1 (F) isolate constructed in this laboratory to contain a single amino acid change in glycoprotein B (gBsyn3) was used in co-infection experiments with the Onc virus to isolate a virus that contained both the Onc and gBSyn3 mutations. The resultant virus (OncSyn) was plaque purified at least six times. The targeted deletions of the γ₁ 34.5 gene and neighboring sequences, including the UL56, α 0, and α 4 genes, and the concomitant insertion of the HcRed gene cassette were confirmed by restriction endonuclease analysis, diagnostic PCR and sequencing. The OncSyn viral genome was recovered as a bacterial artificial chromosome into E. coli. The original pYEbac102 containing the HSV-1 (F) genome was compared to pOncSyn containing the HSV-1 (F) OncSyn genome via restriction EcoRI endonuclease analysis. For diagnostic PCR, primers F-UL54end (A: 5′-AGGAGTGTT CGA GTCGTGTCT-3′ (SEQ ID NO: 1)) and R-ICP4prom (B: 5′-TGGGAC TATATGAGCCCGAG-3′ (SEQ ID NO: 2)) flanking the insertion site were used to confirm the presence of the insertion in place of the deleted genomic region. Primer A mentioned above and primer R-HcRed (C: 5′-CCTGCTGAAG GAGAGTATGCG-3′ (SEQ ID NO: 3)) were used to confirm the presence of the inserted HcRed gene cassette. Primers F-gK (D: 5′-ATGCTCGCCGTCCGTTCCCTGC-3′ (SEQ ID NO: 4)) and R-gK (E: 5′-ATCAAACAG GCGCCTCTGGATC-3′ (SEQ ID NO: 5)) were used to amplify the UL53 gene as a positive control.

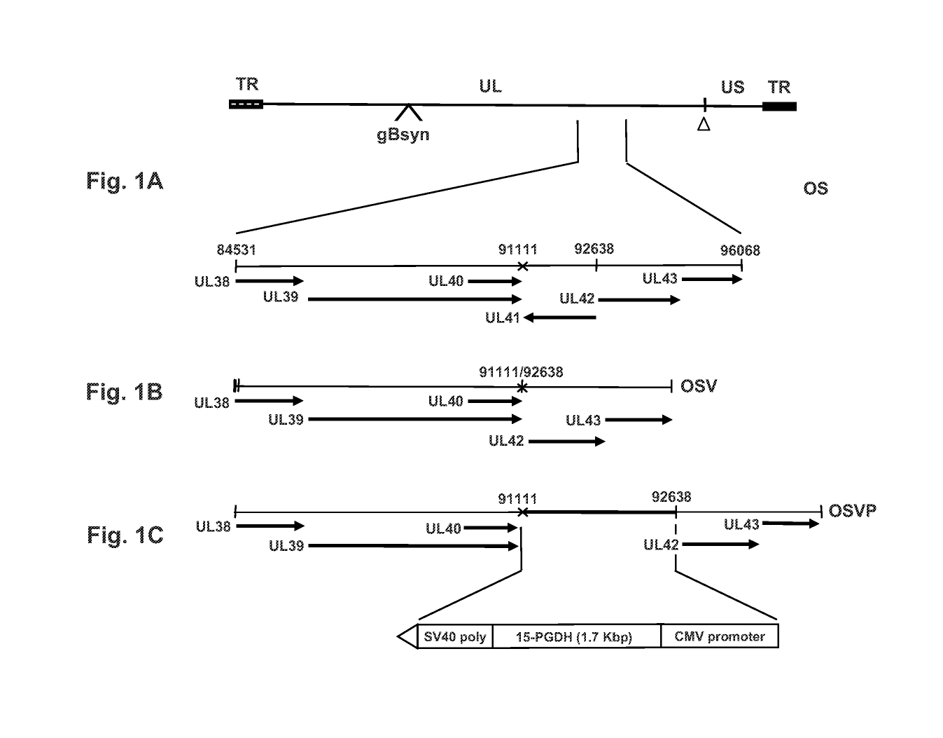

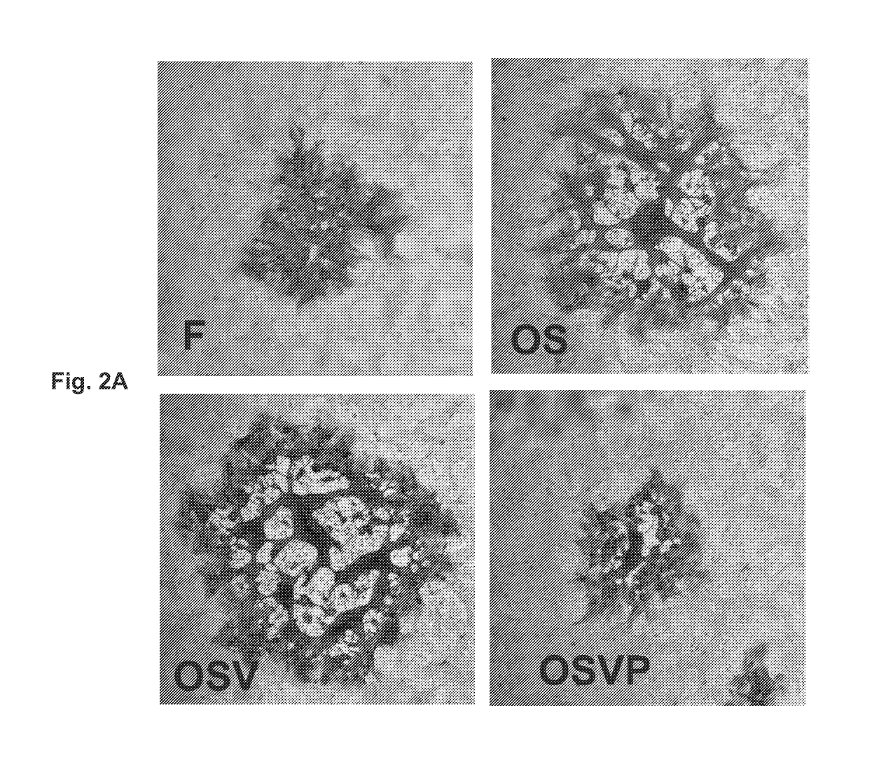

FIG. 1A illustrates the schematic representation of the strategy used to generate the HSV-1 OncSyn (OncSyn; OS) viruses. Specifically, plasmid pRB3410 containing an approximately 16 kilobase pair (Kbp) fragment spanning the viral genomic site containing the γ₁ 34.5 gene was modified to include the HcRed gene cassette (RFP gene under the control of the EF-1α) immediately flanked by the UL54 and a 22 genes. Homologous recombination between the transfer plasmid and the viral genome in a transfection followed by infection experiment resulted in viral plaques emitting red fluorescence when observed under a fluorescence microscope.

To facilitate virus spread via virus-induced cell fusion, the OncSyn virus was isolated after double-infection of Vero cells with Onc and a HSV-1 (F) laboratory strain specifying the gBsyn3 mutation. Individual viral plaques exhibiting the syncytial phenotype and emitting red fluorescence were isolated and extensively plaque-purified. Individual viruses were plaque-purified and the targeted deletion/insertion was verified by DNA sequencing of the entire genomic region bracketing the deletion/insertion, as well as by PCR analyses as described in WO 2008/141151.

### Example 2

### Construction and Testing of OSV and OSVP

### Materials and Methods

Cells.

African green monkey kidney (Vero) cells and mouse mammary tumor cells (4T1) (1) were obtained from the American Type Culture Collection (Manassas, Va.). Vero cells were maintained in Dulbecco's modified Eagle's medium supplemented with 10% fetal bovine serum (FBS) and antibiotics. 4T1 cells and primary mouse cells were maintained in RPMI 1640 medium containing 10% FBS (Invitrogen, Carlsbad, Calif.). The cultures were maintained at 37° C. in a humidified atmosphere of 5% CO₂.

Construction of recombinant OSV and OSVP viruses. The previously published OS viral genome recovered as a bacterial artificial chromosome (bac) in E. coli (pOS) (26; WO 2008/141151, and as discussed above) was used for the construction of pOSV bac plasmid. The coding region of UL41 (nucleotides 91111-92638) was deleted to yield pOSV using double-red mutagenesis in E. coli (68). FIG. 1B illustrates the genome of pOSV. The OSV virus was recovered after transfection of Vero cells with pOSV. pOSVP was generated from pOSV by inserting a ˜3.1 Kbp gene expression cassette containing the Mus 15-musculus hydroxyprostaglandin dehydrogenase (15-NAD) cDNA (cDNA clone MGC:14001 IMAGE:4208980). This vector/clone came with SM40 and CMV promoter as shown in FIG. 1C already attached, although this promoter is probably not necessary. The inserted gene cassette includes nucleotide sequences beginning 12 nucleotides proximal to the Cla I restriction site and extending to 7 nucleotides before the NsiI restriction site of the 15-NAD gene. This fragment was inserted into the locus previously occupied by UL41 in the same genomic orientation as UL41 (right to left), as shown in FIG. 1C. The OSVP virus was recovered after transfection of Vero cells with pOSVP. These modifications were confirmed by restriction enzyme and DNA sequence analyses of the affected genomic regions.

Phenotypic Characterization and Replication Kinetics of the OSV and OSVP Viruses.

Cells (both Vero and 4T1) were seeded into 6-well plates and infected the following day (when they had reached approximately 95% confluency) with either wild-type HSV-1 F strain, OS, OSV or OSVP at a multiplicity of infection (MOI) ranging from 0.001-1 plaque forming units per cell (PFU/cell). Cells were cultured in a maintenance medium (containing 2% FBS and were left for 2 days to allow for cytopathic effects to develop. Infected cells were visualized by immunohistochemistry at 48 hours post-infection (h.p.i.) using horseradish peroxidase-conjugated anti-HSV antibody (Dako, Carpinteria, Calif.) and Novared substrate development kit (VectorLabs, Burlingame, Calif.). Images were captured using phase contrast microscopy, as reported previously. (26)

To determine the replication kinetics of the viruses, one-step growth kinetics and viral titers at 48 h.p.i. were performed as described previously (11, 12). Briefly, nearly confluent monolayers of either Vero or 4T1 cells were infected with each virus at an MOI of 5 at 4° C. for 1 h. Thereafter, virus was allowed to penetrate for 2 h at 37° C. Any remaining extracellular virus was inactivated by low-pH treatment with phosphate buffered saline at pH 3.0. Supernatants were harvested at given time points following normal culture conditions and viral titers were obtained by endpoint titration on Vero cells.

15-PGDH Detection.

Transfection of Vero cells with the 15-PGDH mammalian expression vector was achieved using Lipofectamine 2000 (Invitrogen) according to the manufacturers' suggested protocol. Cell culture supernatant PGE2 levels were determined by ELISA using Prostaglandin E₂ Express EIA Kits (Cayman Chemical, Ann Arbor, Mich.)

4T1 Balb/c Tumor Growth and Pulmonary Metastasis Determinations.

Female Balb/c mice were obtained from Charles River (Wilmington, Mass.) and housed in an animal room which was kept at 25° C. with a 12 hour light-dark cycle. All experimental procedures involving animals were approved by the institutional animal care and use committee (IACUC) of the Louisiana State University. At 6-7 weeks of age, the animals were implanted subcutaneously in the interscapular area with 1×10⁵ viable 4T 1 cells suspended in 0.1 ml of PBS with 20% growth factor reduced Matrigel (BD Biosciences, Franklin Lakes, N.J.) using a 27 gauge needle. Tumor sizes were monitored beginning ˜7 days after tumor inoculation by direct measuring with a digital microcaliper. Tumor volumes were calculated using the following formula: volume=(length×width×height)/2, as reported previously (26). At an average tumor volume of approximately 200 mm³ (12 days post tumor inoculation), animals were randomized into 4 groups. The groups of mice received 3 intratumoral injections of the OS, OSV, OSVP viral particles, or PBS every four days. Each tumor was injected with approximately 1×10⁷ PFU of virus per injection in 0.1 ml volume, while control mice received PBS. Injections were performed slowly at 2 different sites per tumor.

For pulmonary metastatic burden assays, tumor bearing mice treated as above were euthanized with CO₂ at day 29-30 post-tumor inoculation. Lungs from each mouse were excised, minced and digested for 75 minutes at 4° C. while rocking in buffer containing elastase and collagenase type IV. Following filtration through a 100 μm nylon screen, total lung homogenates for each mouse were serially diluted and cultured for 1 week in RPMI 1640 with 10% FBS and 60 μM 6-thioguanine 6-thioguanine resistant colonies were formalin fixed and stained with crystal violet for visualization and counting. Each colony represented one clonogenic metastatic cell (50).

Immunophenotyping of Mouse Splenocytes and Lymphocytes.

All analyses were conducted using freshly harvested splenocytes or draining lymph node cells from animals with similarly sized tumors (150+/−25 mm³). Animals were treated twice (day 1 and day 4) with either PBS, OS, OSV, OSVP, and then spleens and draining lymph nodes were harvested on day 5. The organs were manually dissociated in RPMI and filtered through a 100 μm nylon mesh. Erythrocytes were removed by 5 minute exposure to AKC lysing buffer. Cells for flow cytometry were surface labeled with fluorescently-conjugated antibodies to either murine CD83 (catalog number 558205, BD Biosciences, San Jose, Calif.) to identify activated lymphocytes and dendritic cells, or CD11b and Gr-1 (catalog numbers 552850 and 553126 respectively, BD Biosciences) to identify MDSC. Species-appropriate isotype control antibody conjugates were used in each experiment to identify positive populations (catalog numbers 554685, 552849, 556923 respectively, BD Biosciences). Regulatory T (Treg) cells were labeled using the Mouse Treg Flow Kit from Biolegend, San Diego, Calif. (catalog number 320015) according to the manufacturers' instructions. All cytometric data acquisition was performed on a FACScalibur instrument (BD Biosciences), and analyzed using WinMDI flow cytometry analysis freeware (written by Joseph Trotter, The Scripps Research Institute, La Jolla, Calif.).

For ex vivo effector cytokine production analyses, splenocytes were cultured ex vivo in 96 well format in either control or anti-mouse CD3-coated tissue culture plates (both from BD Biosciences, catalog numbers 354720 and 354730) in triplicate at a density of 100,000 cells per well. Supernatants were harvested after 24 hours incubation and analyzed for murine Th1 and Th2 cytokine production (Bio-Plex Pro Mouse Cytokine TH1/TH2 Assay catalog number M60-00003J7, Bio-Rad, Hercules, Calif.).

Statistical Analyses.

Unless otherwise indicated, Student's t-test was used to determine p values and establish significant differences for sample means (p>0.05). Tumor growth curve analyses used a distribution-free test established for tumor-growth curve comparisons (31). Pulmonary 4T1 metastatic burden comparisons were conducted using the Mann-Whitney non-parametric statistical hypothesis test (p<0.05, one tailed) supplied on GraphPad software (GraphPad Software, Inc., La Jolla, Calif.).

### Example 3

### Construction and characterization of HSV-1 OSVP

The recombinant viruses OSV and OSVP were constructed by double-red mutagenesis of the HSV OS genome cloned into a bacterial artificial chromosome (bac) (26). The OS viral genome lacks a section of the HSV-1 large inverted repeat region encompassing a single copy each of ICP0, γ34.5, and ICP4 genes. This region was replaced by a gene cassette constitutively expressing the red fluorescence protein (RFP; HcRed) under the human cytomegalovirus immediate early promoter (HCMV-IE) control. In addition, the OS genome contains the gBsyn3 syncytial mutation that causes extensive virus-induced cell fusion (FIG. 1A) (25, 26). The double-red recombination method (68) was utilized to delete the entire UL41 ORF (vhs) of the OS bac (bOS) to produce the OSV bac (bOSV) and OSV virus, as described above and as shown in FIG. 1B. The bOSV genome was used for a subsequent round of double-red recombination in which a gene cassette containing the murine 15-PGDH under the control of a CMV-promoter was inserted into the vhs deletion site. The resulting bOSVP genome was transfected into Vero cells to recover the OSVP virus with a genome as illustrated in FIG. 1C. The genotypes of OSV and OSVP viral genomes were confirmed by direct DNA sequencing.

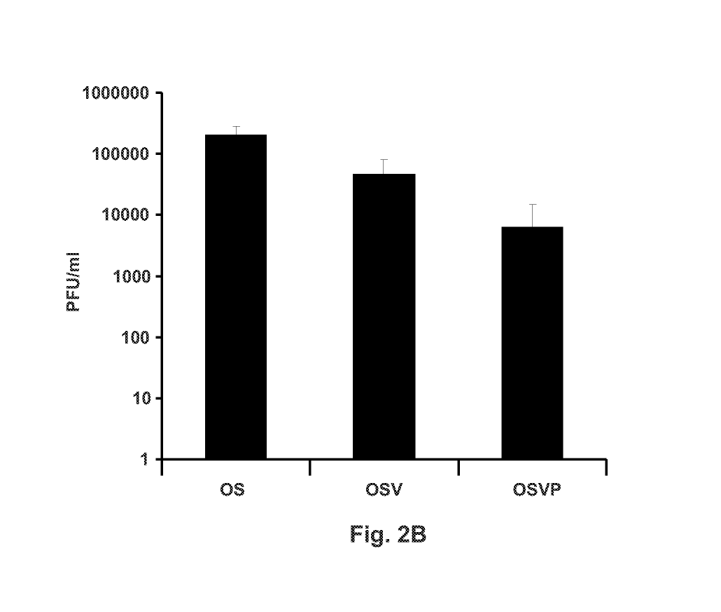

The replication and average plaque morphology characteristics of the OSV and OSVP viruses were compared to their parental virus OS and the prototypic HSV-1 (F) strain used to derive the OS virus in cell culture experiments. The results are shown in FIG. 2A. FIG. 2A shows the morphology and growth of nearly confluent Vero cell monolayers infected with wild-type HSV-1 (F), OS, OSV, or OSVP viruses at MOI of 0.001, as the individual viral plaques were visualized 48 hr post infection by immunohistochemistry and photographed at the same magnification with a phase contrast microscope. The OS virus produced syncytial plaques that were on the average 2-3 times larger than those of the prototypic HSV-1 (F) at 48 hpi. Deletion of the UL41 gene did not appreciably affect plaque morphology characteristics of the resultant OSV virus, in as much as the OSV viral plaques remained syncytial. In contrast, as shown in FIG. 2A, the OSV and OSVP viruses produced viral plaques that on average were smaller than those of the OS virus. However, OSVP produced syncytial plaque phenotypes (FIG. 2A) and caused extensive virus-induced cell fusion especially in high MOI infections of Vero cell monolayers (data not shown).

The ability of the OS, OSV and OSVP viruses to replicate in 4T1 cells was examined by determining viral titers obtained at 48 hpi infection with an MOI of 5. These results are shown in FIG. 2B. FIG. 2B shows the viral titers (pfu/ml) for triplicate cultures collected at 48 hr post infection of nearly confluent 4T1 cell monolayers infected with wild-type HSV-1 (F), OS, OSV, or OSVP viruses at an MOI of 5. OSVP produced viral titers that were approximately one-log lower than the OS virus, while OSV produced intermediate titers approximately 5-fold lower than those of the OS virus. Additional experiments in Vero cells revealed a similar virus production pattern with OSVP producing consistently approximately one-log less virus than the OS virus (data not shown).

Deletion of the vhs gene did not drastically affect virus replication in Vero or mouse 4T1 cells, or virus-induced cell fusion caused by the gBsyn3 syncytial mutation of the parental OS virus. The vhs-deleted OSV genome contains an approximately 11 kbp deletion encompassing the UL-US junction and the UL41(vhs) deletion (2.5 kbp) for a total of approximately 13.5 kbp deleted sequences. Therefore, this virus is amenable to insertion of multiple gene cassettes expressing genes of interest to cancer therapy without exceeding viral DNA packaging limits.

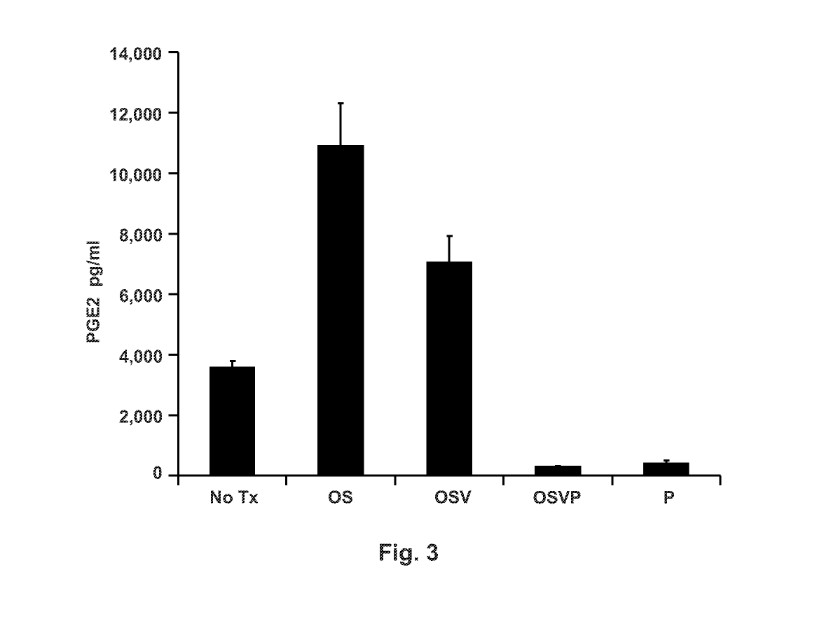

The 15-PGDH gene cassette was inserted in-place of the vhs deletion on the OSV viral genome cloned as a bacterial artificial chromosome (bac). The OSVP-bac (bOSVP) was stable in E. coli and did not contain any unintended genetic alterations, as evidenced by restriction endonuclease analysis and DNA sequencing (data not shown). The OSVP virus appeared to replicate less efficiently (5-fold less) than either the OS and OSV viruses, although the syncytial phenotype of the virus was unaffected. Expression of 15-PGDH by OSVP was expected and was shown (FIG. 3, below) to decrease intratumor PGE2 levels potentially resulting in lower viral growth. However, 15-PGDH expression did not drastically reduce viral replication in cell culture (FIG. 2), or within tumors (not shown) suggesting that basal levels of PGE2 remaining in tumors after PGDH expression are sufficient for viral replication.

A critical consideration in the use of oncolytic herpes viruses in human cancer therapy is the provision for multiple safety measures against uncontrolled propagation of the virus in the host. The parental OS virus carries a large deletion encompassing the UL-US junction and containing one copy of the ICP34.5 gene, as well as immediate early genes ICP0, ICP4 and one of the two LAT gene loci in the viral genome. This deletion is similar to the deletion carried by the NV1020 virus that has been extensively tested in pre-clinical animal models and recently in human studies showing a high safety profile. The vhs gene is known to be critically important for viral pathogenesis. Specifically, deletion of the vhs gene substantially reduced ability of the virus to grow in trigeminal ganglia, brains and cornea. Deletion of the vhs gene provides for significant increase in viral attenuation and safety. Production of 15-PGDH provides for an additional level of safety, since high levels of PGE2 increase viral replication and virus spread and reactivation from latency.

### Example 4

### OSVP Inhibits PGE2 Accumulation

To evaluate the ability of the OSVP virus to produce functional 15-PGDH, 4T1 cells were infected with OSVP virus at an MOI of 5. At 48 hpi supernatants of the infected cells were collected and assayed for cumulative levels of PGE2. The results are shown in FIG. 3. FIG. 3 illustrates the level of PGE2 as measured in nearly confluent monolayers of 4T1 cells infected with wild-type HSV-1 (F), OS, OSV, or OSVP viruses at a MOI of 5, and harvested and assayed for PGE2 two days post infection by ELISA immunoassay. In FIG. 3, “P” denotes transient expression with a 15-PGDH mammalian expression vector, while “No Tx” denotes mock-treated cells. PGE2 assays were performed in triplicates and error bars represent the 95% confidence interval (CI) of the mean. 4T1 cells infected with OSVP, or transfected with the 15-PGDH expression vector alone displayed significantly reduced levels of supernatant PGE2 relative to the no treatment control (>10 fold). In contrast, infection with OS or OSV resulted in significant increases in supernatant PGE2 (FIG. 3).

### Example 5

### OSV and OSVP Inhibit 4T1 Tumor Growth

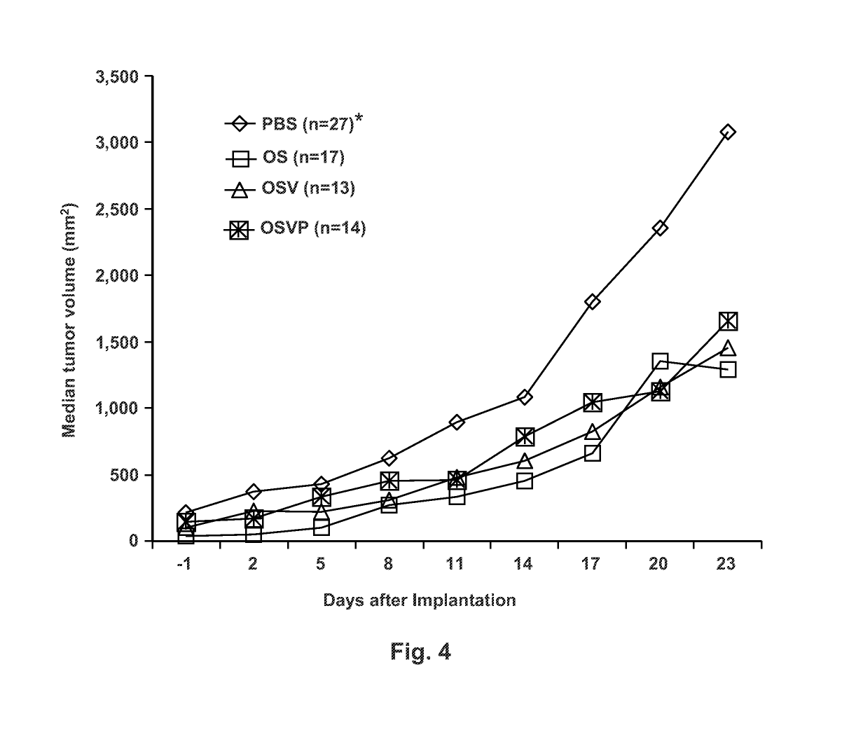

Previously, treatment of 4T1 tumors in immunocompetent Balb/c mice with the OS virus was shown to result in substantial reduction in tumor growth and metastasis to the lung and other organs (26). Similar experiments were performed to assess the relative abilities of the OSV and OSVP virus to inhibit 4T1 tumors. Mice bearing palpable tumors were treated with four doses of virus (1×10⁷ PFU/dose) injected intratumorally at 3 day intervals. FIG. 4 illustrates the change in tumor volume, measured using a digital caliper at defined time intervals prior and after treatment (X axis), in Balb/c mice implanted subcutaneously in the interscapular area with 1×10⁵ viable 4T1 cells. When tumors reached approximately 80-90 mm³ in volume, the tumors were injected with each virus (1×10⁷ PFU/ml in PBS buffer), or PBS alone. Tumors were treated with each of the OS, OSV, or OSVP or control at days 1, 3 and 6. The asterisk indicates statistical significance by non-parametric analysis. The results shown are from one of three independent experiments that produced similar results. Mice treated similarly with PBS served as negative controls. Tumor measurements were collected until the mice were sacrificed at day 20 (post treatment). All three viruses, OS, OSV and OSVP, resulted in a similar reductions of tumor growth as compared with the control (FIG. 4).

### Example 6

### OSV and OSVP Treatments Reduce the Incidence of Pulmonary Metastases

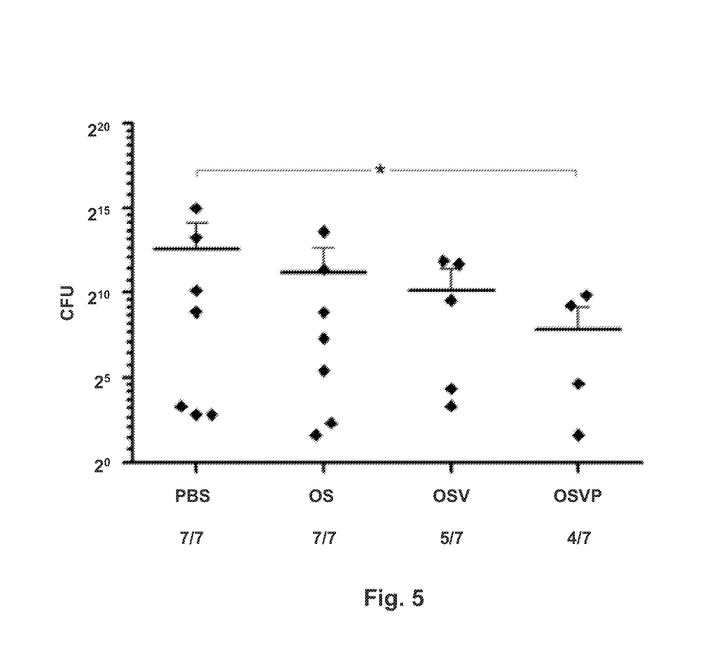

Previously, OS treatment of 4T1 tumors in Balb/c mice was shown to reduce the incidence of tumor metastases (25). To compare the relative abilities of OSV and OSVP viruses to inhibit tumor metastasis, pulmonary metastatic burdens were assessed in treated and untreated animals 30 days post-tumor inoculation, by determining the number of 6-thioguanine-resistent clonogenic 4T1-cells per mouse (see Example 1, Materials and Methods). FIG. 5 illustrates the metastatic loads calculated by the total pulmonary clonogenic metastatic foci enumerated by limited dilution culture in the presence of 6-thioguanine from lungs harvested from virus-treated mice bearing 4T1 tumors that were sacrificed 30 days post tumor inoculation. The metastasis incidence rate for each group is indicated below the x-axis. Means for each group are represented by the horizontal bars with error bars delineating 95% CI. The results shown are from one of three independent experiments that produced similar results. OSVP-treatment significantly reduced the pulmonary metastatic burden in comparison to the PBS-treated control. In contrast, OS and OSV treatments resulted in intermediate reductions in metastatic pulmonary tumors (FIG. 5).

### Example 7

### OSVP Alleviates PGE2-Based Immune Suppression in Mice with Advanced 4T1 Tumors

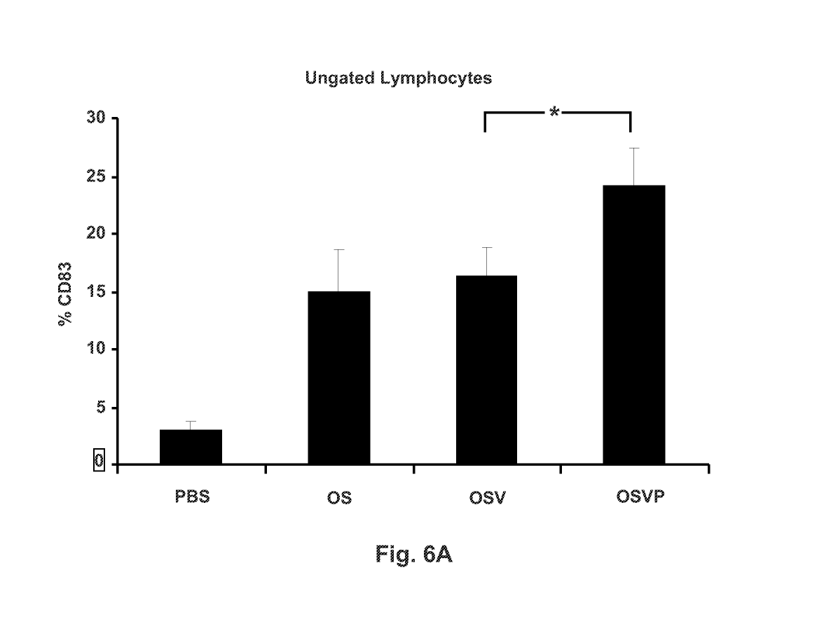

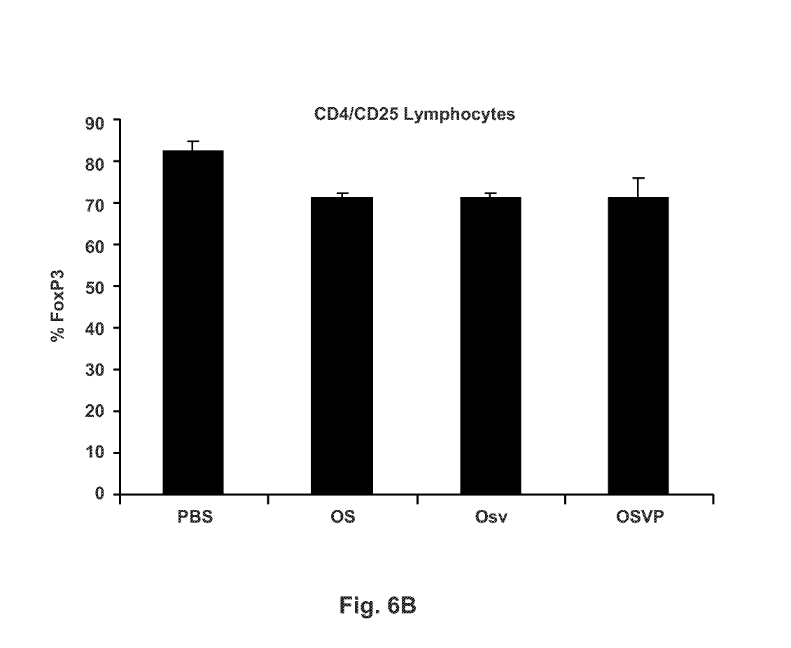

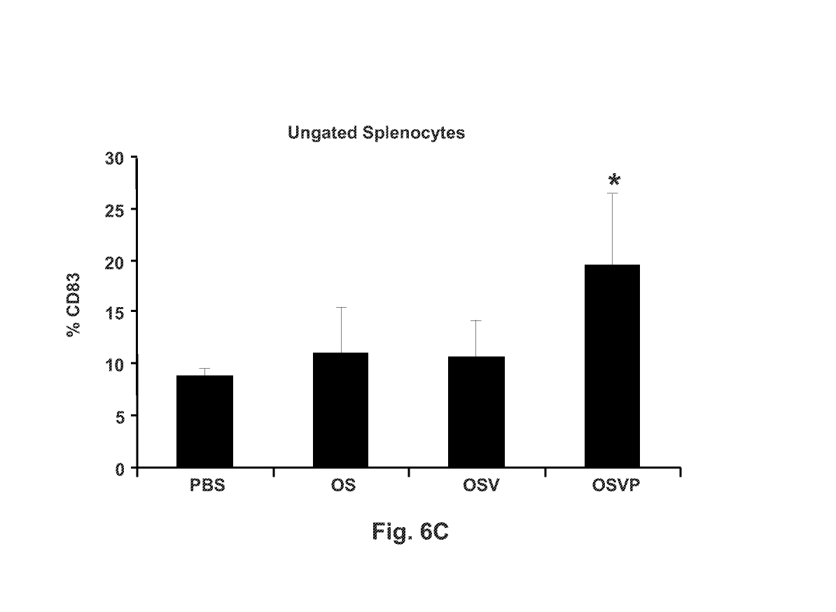

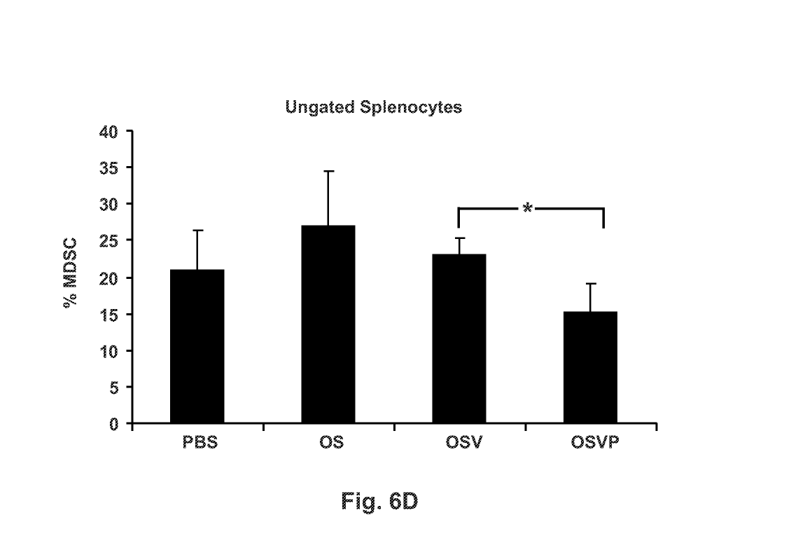

PGE2 promotes the development and activity of immunosuppressive cell populations including MDSC and Treg (53, 57, 58). To examine the effect of 15-PGDH expression and expected PGE2 reduction on immune suppression in mice treated with the OSVP virus, the relative proportions of splenocytes and lymphocytes of lymph node origin possessing suppressor cell phenotypes were determined by polychromatic flow cytometry. FIGS. 6A-6D illustrates the immune response in 4T1 tumor bearing mice with similar-sized, and well developed tumors measured in cells isolated two days after being given a single intratumoral injection of the control (PBS) or of the OS, OSV, or OSVP viruses. FIGS. 6A-6B illustrate specific immunogenic cells from draining lymph nodes with FIG. 6A showing the CD83 activation marker, and FIG. 6B showing the CD4+CD25+FoxP3+regulatory T cells. FIGS. 6C and 6D illustrate specific immunogenic markers isolated from splenocytes with FIG. 6C showing the CD83 activation marker, and FIG. 6D showing the MDSC markers (Gr1⁺, CD11b⁺). Indicated phenotypes are shown as the mean percentage of total live cells. Error bars represent 95% CI of the means, and n=3 unless otherwise noted.

OSVP-treated mice exhibited a significant decrease in splenic MDSC compared to OSV (FIG. 6D. In contrast, splenic Tregs were not significantly reduced (data not shown), although oncolytic therapy alone appeared sufficient to inhibit some Treg accumulation in lymphocytes from lymph nodes (FIG. 6B). The proportions of CD83-positive cells (representing mature DC and activated T cells) present in spleens from OSVP-treated mice were significantly increased relative to PBS, OS or OSV treated controls (FIG. 6C). Similarly, CD83+lymphocytes were significantly increased in OSVP-treated animals in comparison to PBS, OS and OSV-treated controls (FIG. 6A).

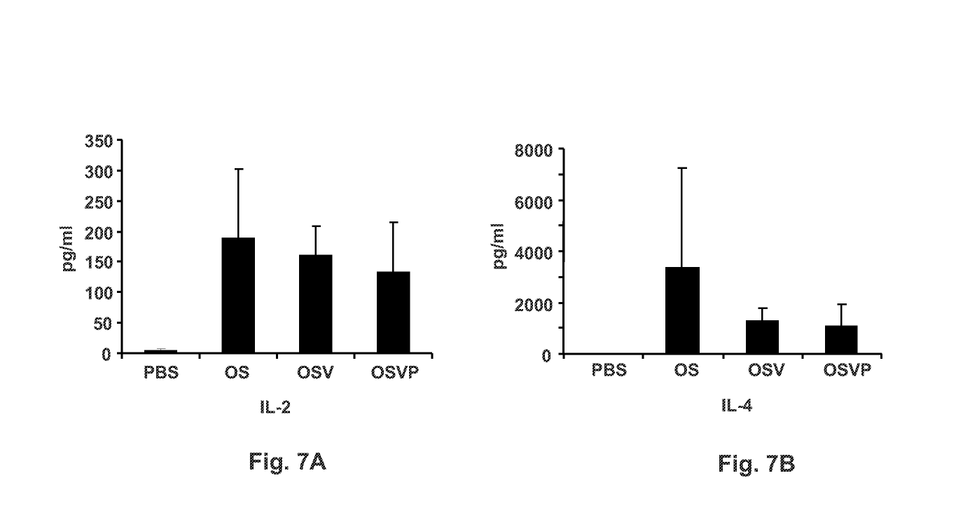

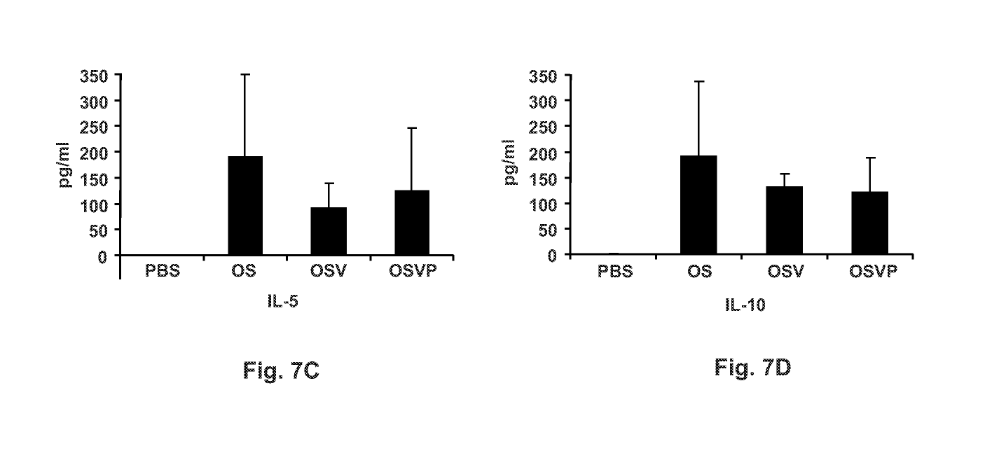

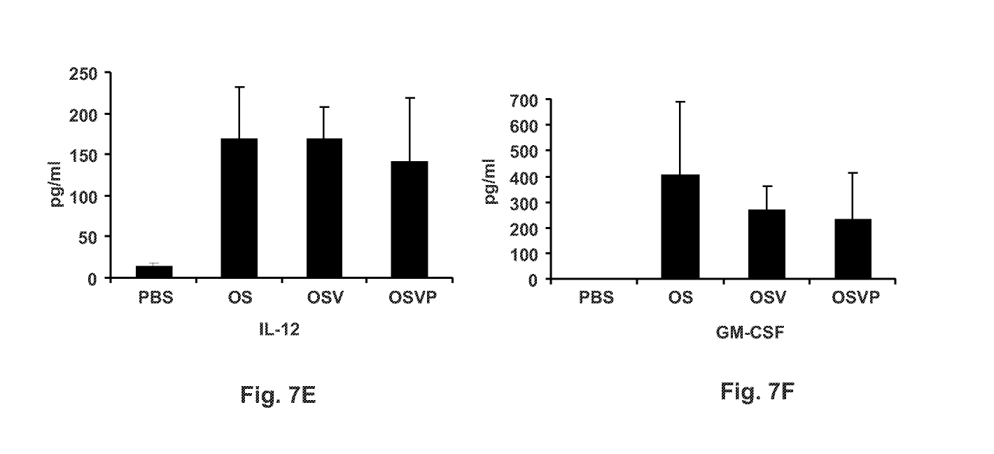

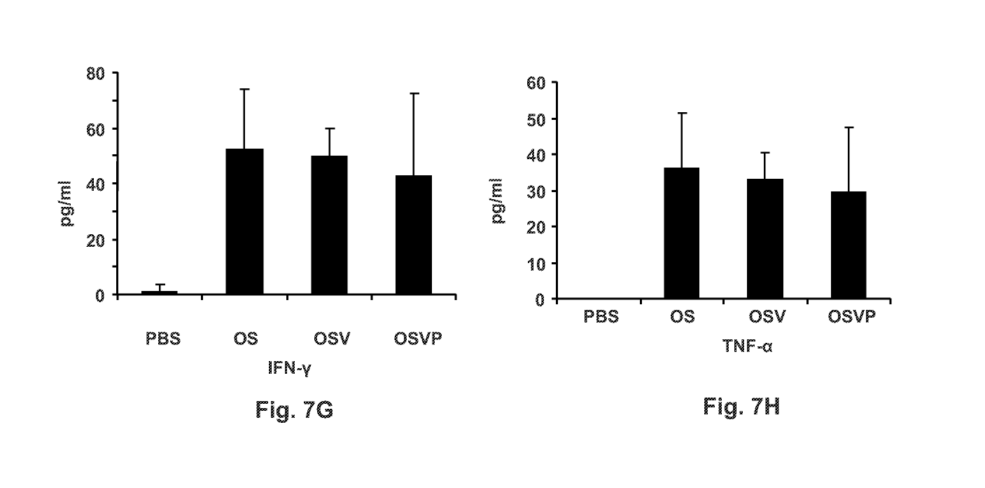

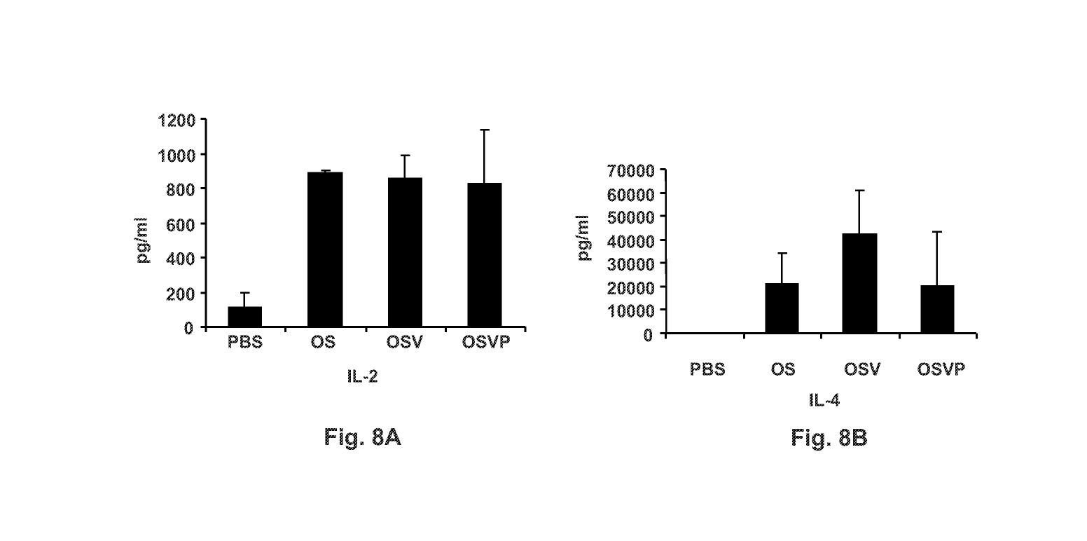

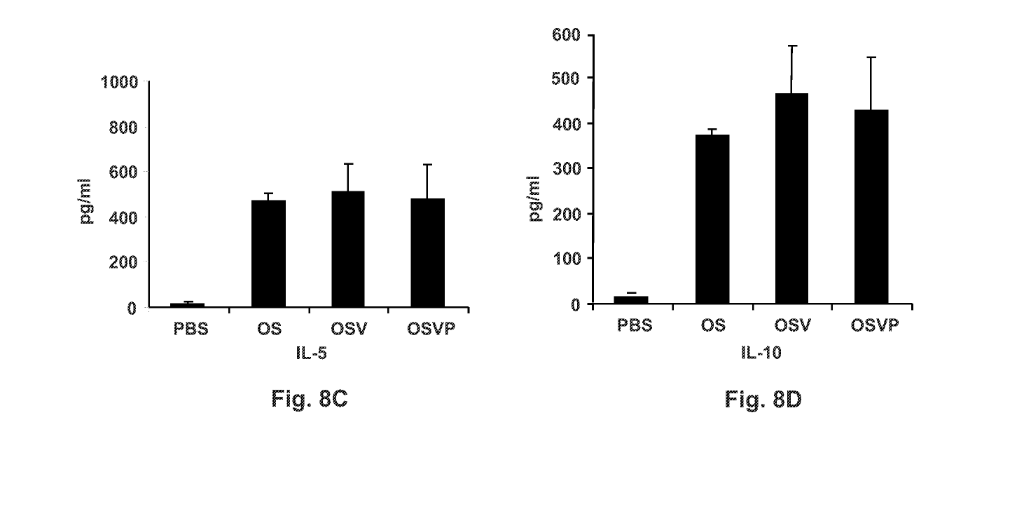

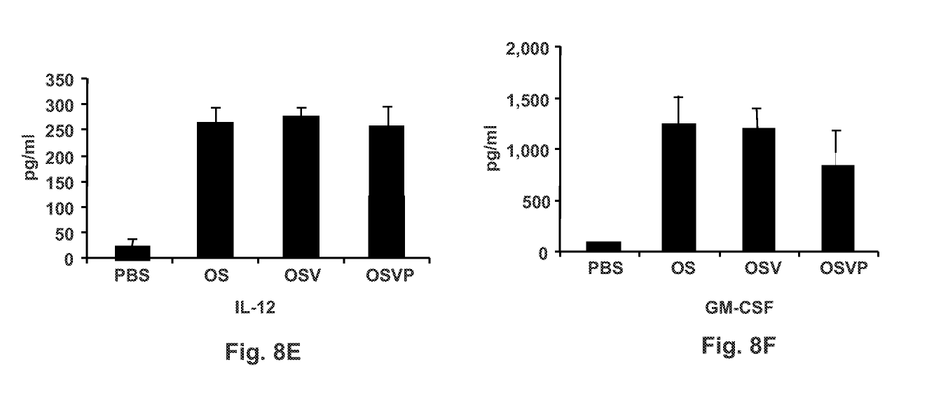

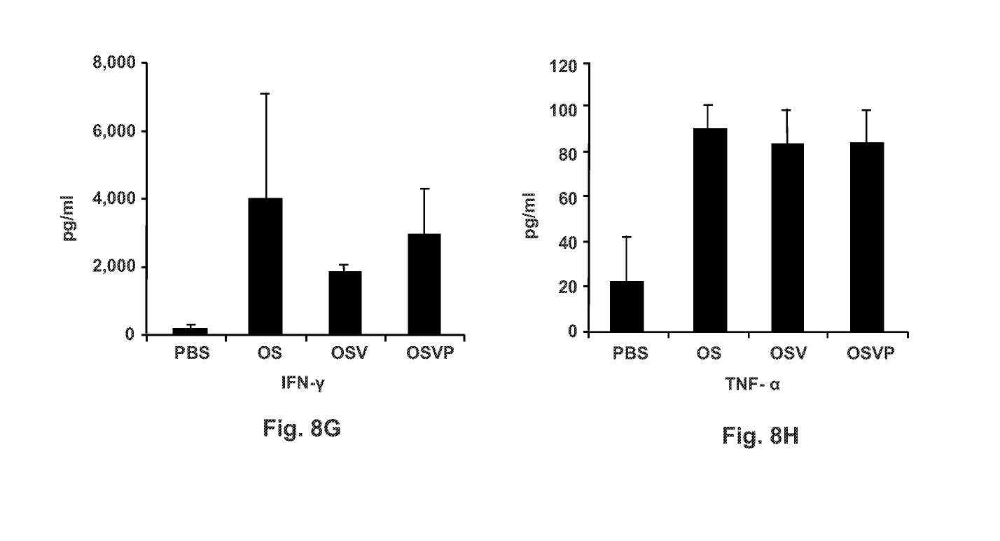

Th1/Th2 cytokine profiling was performed to assess baseline and stimulated immune activity of splenocytes from treated and control mice bearing similar size tumors. FIGS. 7A-7H and 8A-8H illustrate the cytokine immunoprofiles of lymphocytes from draining lymph nodes derived from mice with similarly-sized 4T1 tumors (200-300 mm²) that were treated twice with a 3 day interval with OS, OSV, OSVP or PBS (control), and isolated two days after the second treatment. The lymphocytes were isolated and cultured either alone (FIGS. 7A-7H), or with immobilized anti-CD3 stimulatory antibody (FIGS. 8A-8H) at a concentration of 100,000 cells per ml. After culturing for 24 hours, supernatants were assayed for TH1/TH2 cytokine production by Bioplex (Bio-Rad). Shown are the mean quantities of the indicated cytokines in pg/ml with error bars representing 95% CI (n=3).

Generally, OS, OSV and OSVP viruses appeared to stimulate unbiased effector helper T cell activity, as evidenced by the production of similar levels of Th1 (IL-2, GM-CSF, IFN-γ, TNF-α) and Th2 (IL-5, IL4, IL-10, IL-12) in comparison to PBS-treated control animals (FIGS. 7A-7H). Polyclonal stimulation of isolated splenocytes with immobilized CD3 antibody produced a similar pattern of immune stimulation to that of unstimulated splenocytes, with the exception that a significantly more robust production of the relevant cytokines was observed in comparison to PBS-treated animals (FIG. 8A-8H).

Th1/Th2 cytokine profiling revealed that all three viruses (OS, OSV and OSVP) stimulated the production of similar levels of both Th1 (IL-2, GM-CSF, IFN-γ, TNF-α) and Th2 (IL-5, IL4, IL-10, IL-12) cytokines. These results suggest that although the absence of the vhs protein and constitutive expression of 15-PGDH may directly or indirectly affect dendritic cell functions and maturation, these effects did not appreciably alter the potent local and systemic immunostimulatory properties of these viruses. However, splenic effector cell responses to polyclonal T cell activation were considerably enhanced in treated animals. Moreover, the presence of increased CD83 positive cells in draining lymph nodes of OSVP-treated tumors suggests that OSVP may provide additional immune enhancement. This robust immune stimulation will be expected to increase the generation of anti-tumor immune responses. Importantly, lack of the vhs gene may allow increased expression of tumor-associated antigens (TAA) into the tumor microenvironment for uptake by dendritic cells leading to adaptive T cell priming. On the other hand, 15-PGDH expression may help ensure the overall viability of dendritic cells for optimum TAA presentation by dendritic and other professional antigen-presenting cells.

CD11b and GR-1 double positive cells (MDSC) and Tregs, indicated by CD3, FoxP3, and CD25 staining represent a diverse population of PGE2 sensitive immune cells with suppressor functions that are induced by many cancers including 4T1. The OSVP virus substantially reduced the relative numbers of MDSCs in treated animals in comparison to either OS or OSV viruses suggesting that the observed MDSC inhibition was caused by the 15-PGDH expression and the concomitant reduction in PGE2 levels. The observed MDSC reduction in peripheral lymphocyte and splenocyte populations suggest that intratumor expression of 15-PGDH is capable of producing systemic reduction of MDSCs. Similar results have been obtained after adenovirus-mediated delivery of 15-PGDH in CT-26 colon carcinomas implanted in mice. This work showed not only reduction of MDSCs, but also the differentiation of intratumoral CD11b cells from immunosuppressive phenotypes to MHC class II-positive myeloid APCs (10).

OSVP treatment of highly metastatic 4T1 mouse breast tumors resulted in substantial reduction of metastasis to mouse lungs. Unlike the adenovirus vector with PGDH (10), the OSVP oncolytic vector platform holds particular promise for cancer therapy because it can accommodate the simultaneous expression of additional genes that can stimulate anti-tumor immune responses and alter the immunosuppressive milieu in the tumor microenvironment. In addition, the virus can accommodate the heterologous expression of specific TAA that can potentially augment antigen-specific anti-tumor immune responses.

### Example 8

### Combination Therapy of OSVP and a Cancer Drug or a NSAID

Taxanes, like paclitaxel and docetaxel are microtubule stabilizers that kill dividing cancer cells. We will assess the anti-tumor effects of combined OSVP/paclitaxel treatment on 4T1-tumor bearing immune-competent mice. Paclitaxel will be administered intraperitoneally beginning approximately two days before the onset of OSVP treatment and continued throughout the study at 2 day intervals. Untreated and single therapy (i.e., treated only with paclitaxel or OSVP) animals will be used as controls. It is anticipated that enhanced tumor-killing by taxane co-therapy in the presence of lowered PGE2 generated by OSVP will promote improved anti-tumor immunity and decrease tumor growth and metastasis.

Over-the-counter and prescription anti-inflammatory drugs known as non-steroidal anti-inflammatory drugs (NSAIDs) have been safely used for many decades to alleviate pain and inflammation. We will test the ability of ibuprofen, a non-selective COX2 inhibitor, to augment the therapeutic effects of OSVP treatment. Since OSVP alone reduces PGE2, we anticipate that more substantial reductions will be achieved with the help of an anti-inflammatory drug, and thereby permit the development of more potent anti-tumor immunity. The effects of combination therapy on tumor-derived immune suppression will be examined by measuring pulmonary tumor burdens in treated mice. We will test whether pharmacologically decreased PGE2 correlates with improved OSVP-treatment efficacy and anti-tumor immunity by supplementing viral therapy with continuous oral administration of low-dose NSAIDs.

OSVP-infected 4T1 cells will be cultured in the presence of varying doses of either ibuprofen or paclitaxel to determine the effects of these drugs on OSVP replication. OSVP infection of treated cells will be monitored by determining supernatant virus titers by plaque assay on Vero cells.

Ten six week-old female Balb/c mice (Charles River) per treatment group (BS, drug alone, OSVP alone, drug, and OSVP) will be injected on one side of the back, subcutaneously and caudal to the scapula with 1×10⁵ 4T1 cells (ATCC# CRL-2539) suspended in 100 μl PBS. For combination therapies, children's ibuprofen (commercially available over the counter) will be orally administered daily during viral treatments and injectable paclitaxel will be delivered intraperitoneally (50 mg/kg) every 2 days beginning on day 5 post tumor inoculation. OSVP treatments will begin when palpable tumors (2-5 mm diameter) develop after about 8 days post tumor inoculation and 4-5 days following the initiation of drug therapy. Tumor measurements will be taken at 3 day intervals using digital microcalipers. Three measurements will be taken of each tumor dimension, the product of which will be divided by 2 to establish tumor volume, as described above in Example 1. At the end of the study period, when the maximum tumor dimension reaches 2 cm (approximately 4 weeks), the mice will be humanely sacrificed, and the lungs will be harvested for metastasis assays (see below). To maximize statistical power, these experiments will be repeated two more times. Tumor growth inhibition will be determined by comparison of median tumor growth curves.

Clonogenic Metastasis Assay:

Lungs will be harvested from tumor bearing mice at 20-30 days post inoculation. Following physical and enzymatic dissolution of the tissue, serial dilutions will be plated in 6 well tissue culture plastic and cultured for 14 days in the presence of 6-thioguanine. After incubation, colonies of 4T1 cells will be fixed and stained for visualization and counting. Colonies with >50 cells will be counted as metastatic foci and used to calculate total lung metastatic foci concentrations per mouse.

Synergistic interactions between cancer treatments have been a significant boon to cancer patients. By lowering effective doses of each drug, debilitating toxicities can be avoided leading to longer and more frequent treatments, better prognoses, and improved quality of life for many cancer patients. The promise of oncolytic virotherapy as an alternative/supplement to toxic drug treatments is only increased by the potential for synergistic interaction with other kinds of therapy. Given its ability to reduce PGE2 levels and its nature as an oncolytic agent, it is predicted that OSVP will be particularly sensitive to the effects of NSAIDs. Moreover, the synergistic cancer-killing effects of taxane or angiostatic co-therapy with oncolytic herpes may further reduce tumor-based immune suppression and permit more effective anti-tumor immunity to develop.

### Example 9

### OSVP Inhibits Prostate Tumor Growth

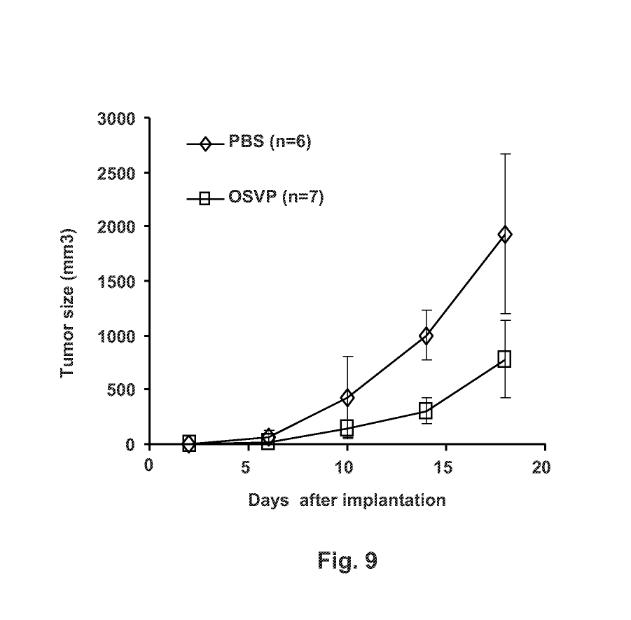

Experiments were performed to assess the ability of the OSVP virus to inhibit RM-9 prostate cancer tumors. Mice bearing palpable tumors were treated with four doses of the OSVP virus (1×10⁶ PFU/dose) injected intratumorally at 4 day intervals. FIG. 9 illustrates the change in tumor volume over time (days), measured using a digital caliper at defined time intervals prior and after treatment (X axis), in male syngeneic C57BL/6 mice implanted subcutaneously on the dorsum with 0.5×10⁶ viable RM-9 prostate cancer cells, and then the tumors were injected with either control or the OSVP virus (1×10⁶ PFU/ml in PBS buffer) when tumors reached approximately 80-90 mm³ in volume. The RM-9 cells and mice were supplied by Dr. Inder Sehgal, Louisiana State University, School of Veterinary Medicine. Tumors were treated with PBS or OSVP at days 2, 6, and 10. Shown are the mean tumor sizes with error bars representing 95% CI. Tumor measurements were collected until the mice were sacrificed at day 18 (post treatment). Treatment with OSVP resulted in a significant reduction in tumor growth as compared with the control (FIG. 9).

### REFERENCES

1. Aslakson, C. J., and F. R. Miller. 1992. Selective events in the metastatic process defined by analysis of the sequential dissemination of subpopulations of a mouse mammary tumor. Cancer Res 52:1399-405.
2. Backlund, M. G., J. R. Mann, V. R. Holla, F. G. Buchanan, H. H. Tai, E. S. Musiek, G. L. Milne, S. Katkuri, and R. N. DuBois. 2005. 15-Hydroxyprostaglandin dehydrogenase is down-regulated in colorectal cancer. J Biol Chem 280:3217-23.
3. Becker, Y., E. Tavor, Y. Asher, C. Berkowitz, and M. Moyal. 1993. Effect of herpes simplex virus type-1 UL41 gene on the stability of mRNA from the cellular genes: beta-actin, fibronectin, glucose transporter-1, and docking protein, and on virus intraperitoneal pathogenicity to newborn mice. Virus Genes 7:133-43.
4. Bennett, J. J., K. A. Delman, B. M. Burt, A. Mariotti, S. Malhotra, J. Zager, H. Petrowsky, S. Mastorides, H. Federoff, and Y. Fong. 2002. Comparison of safety, delivery, and efficacy of two oncolytic herpes viruses (G207 and NV1020) for peritoneal cancer. Cancer Gene Ther 9:935-45.
5. Chou, J., E. R. Kern, R. J. Whitley, and B. Roizman. 1990. Mapping of herpes simplex virus-1 neurovirulence to gamma 134.5, a gene nonessential for growth in culture. Science 250:1262-6.
6. Chou, J., and B. Roizman. 1992. The gamma 1(34.5) gene of herpes simplex virus 1 precludes neuroblastoma cells from triggering total shutoff of protein synthesis characteristic of programed cell death in neuronal cells. Proc Natl Acad Sci USA 89:3266-70.
7. Cotter, C. R., M. L. Nguyen, J. S. Yount, C. B. Lopez, J. A. Blaho, and T. M. Moran. The virion host shut-off (vhs) protein blocks a TLR-independent pathway of herpes simplex virus type 1 recognition in human and mouse dendritic cells. PLoS One 5:e8684.
8. Cozzi, P. J., S. Malhotra, P. McAuliffe, D. A. Kooby, H. J. Federoff, B. Huryk, P. Johnson, P. T. Scardino, W. D. Heston, and Y. Fong. 2001. Intravesical oncolytic viral therapy using attenuated, replication-competent herpes simplex viruses G207 and Nv1020 is effective in the treatment of bladder cancer in an orthotopic syngeneic model. FASEB J 15:1306-8.
9. Ding, Y., M. Tong, S. Liu, J. A. Moscow, and H. H. Tai. 2005. NAD+-linked 15-hydroxyprostaglandin dehydrogenase (15-PGDH) behaves as a tumor suppressor in lung cancer. Carcinogenesis 26:65-72.
10. Eruslanov, E., S. Kaliberov, I. Daurkin, L. Kaliberova, D. Buchsbaum, J. Vieweg, and S. Kusmartsev. 2009. Altered expression of 15-hydroxyprostaglandin dehydrogenase in tumor-infiltrated CD11b myeloid cells: a mechanism for immune evasion in cancer. J Immunol 182:7548-57.
11. Foster, T. P., X. Alvarez, and K. G. Kousoulas. 2003. Plasma membrane topology of syncytial domains of herpes simplex virus type 1 glycoprotein K (gK): the UL20 protein enables cell surface localization of gK but not gK-mediated cell-to-cell fusion. J Virol 77:499-510.
12. Foster, T. P., G. V. Rybachuk, and K. G. Kousoulas. 2001. Glycoprotein K specified by herpes simplex virus type 1 is expressed on virions as a Golgi complex-dependent glycosylated species and functions in virion entry. J Virol 75:12431-8.
13. Fukuhara, H., and T. Todo. 2007. Oncolytic herpes simplex virus type 1 and host immune responses. Curr Cancer Drug Targets 7:149-55.
14. Gabrilovich, D. 2004. Mechanisms and functional significance of tumour-induced dendritic-cell defects. Nat Rev Immuno14:941-52.
15. Geevarghese, S. K., D. A. Geller, H. A. de Haan, M. Horer, A. E. Knoll, A. Mescheder, J. Nemunaitis, T. R. Reid, D. Y. Sze, K. K. Tanabe, and H. Tawfik. Phase I/II study of oncolytic herpes simplex virus NV 1020 in patients with extensively pretreated refractory colorectal cancer metastatic to the liver. Hum Gene Ther 21:1119-28.
16. Geiss, B. J., T. J. Smith, D. A. Leib, and L. A. Morrison. 2000. Disruption of virion host shutoff activity improves the immunogenicity and protective capacity of a replication-incompetent herpes simplex virus type 1 vaccine strain. J Virol 74:11137-44.
17. Greenhough, A., H. J. Smartt, A. E. Moore, H. R. Roberts, A. C. Williams, C. Paraskeva, and A. Kaidi. 2009. The COX-2/PGE2 pathway: key roles in the hallmarks of cancer and adaptation to the tumour microenvironment. Carcinogenesis 30:377-86.
18. Gutermann, A., E. Mayer, K. von Dehn-Rothfelser, C. Breidenstein, M. Weber, M. Muench, D. Gungor, J. Suchnel, U. Moebius, and M. Lechmann. 2006. Efficacy of oncolytic herpesvirus NV 1020 can be enhanced by combination with chemotherapeutics in colon carcinoma cells. Hum Gene Ther 17:1241-53.
19. Harbour, D. A., W. A. Blyth, and T. J. Hill. 1978. Prostanglandins enhance spread of herpes simplex virus in cell cultures. J Gen Virol 41:87-95.
20. Harizi, H., M. Juzan, C. Grosset, M. Rashedi, and N. Gualde. 2001. Dendritic cells issued in vitro from bone marrow produce PGE(2) that contributes to the immunomodulation induced by antigen-presenting cells. Cell Immunol 209:19-28.
21. Harris, R. E. 2007. Cyclooxygenase-2 (cox-2) and the inflammogenesis of cancer. Subcell Biochem 42:93-126.
22. Harris, R. E., J. Beebe-Donk, and G. A. Alshafie. 2007. Cancer chemoprevention by cyclooxygenase 2 (COX-2) blockade: results of case control studies. Subcell Biochem 42:193-212.
23. Harris, R. E., J. Beebe-Donk, H. Doss, and D. Burr Doss. 2005. Aspirin, ibuprofen, and other non-steroidal anti-inflammatory drugs in cancer prevention: a critical review of non-selective COX-2 blockade (review). Oncol Rep 13:559-83.
24. Harris, S. G., J. Padilla, L. Koumas, D. Ray, and R. P. Phipps. 2002. Prostaglandins as modulators of immunity. Trends Immunol 23:144-50.
25. Israyelyan, A., V. N. Chouljenko, A. Baghian, A. T. David, M. T. Kearney, and K. G. Kousoulas. 2008. Herpes simplex virus type-1 (HSV-1) oncolytic and highly fusogenic mutants carrying the NV1020 genomic deletion effectively inhibit primary and metastatic tumors in mice. Virol J 5:68.
26. Israyelyan, A. H., J. M. Melancon, L. G. Lomax, I. Sehgal, C. Leuschner, M. T. Kearney, V. N. Chouljenko, A. Baghian, and K. G. Kousoulas. 2007. Effective treatment of human breast tumor in a mouse xenograft model with herpes simplex virus type 1 specifying the NV 1020 genomic deletion and the gBsyn3 syncytial mutation enabling high viral replication and spread in breast cancer cells. Hum Gene Ther 18:457-73.
27. Kaliberova, L. N., S. A. Kusmartsev, V. Krendelchtchikova, C. R. Stockard, W. E. Grizzle, D. J. Buchsbaum, and S. A. Kaliberov. 2009. Experimental cancer therapy using restoration of NAD+-linked 15-hydroxyprostaglandin dehydrogenase expression. Mol Cancer Ther 8:3130-9.
28. Kelly, K. J., J. Wong, and Y. Fong. 2008. Herpes simplex virus NV1020 as a novel and promising therapy for hepatic malignancy. Expert Opin Investig Drugs 17:1105-13.
29. Kemeny, N., K. Brown, A. Covey, T. Kim, A. Bhargava, L. Brody, B. Guilfoyle, N. P. Haag, M. Karrasch, B. Glasschroeder, A. Knoll, G. Getrajdman, K. J. Kowal, W. R. Jarnagin, and Y. Fong. 2006. Phase I, open-label, dose-escalating study of a genetically engineered herpes simplex virus, NV 1020, in subjects with metastatic colorectal carcinoma to the liver. Hum Gene Ther 17:1214-24.
30. Khyatti, M., and J. Menezes. 1990. The effect of indomethacin, prostaglandin E2 and interferon on the multiplication of herpes simplex virus type 1 in human lymphoid cells. Antiviral Res 14:161-72.
31. Koziol, J. A., D. A. Maxwell, M. Fukushima, M. E. Colmerauer, and Y. H. Pilch. 1981. A distribution-free test for tumor-growth curve analyses with application to an animal tumor immunotherapy experiment. Biometrics 37:383-90.
32. Kwong, A. D., and N. Frenkel. 1987. Herpes simplex virus-infected cells contain a function(s) that destabilizes both host and viral mRNAs. Proc Natl Acad Sci USA 84:1926-30.
33. Kwong, A. D., J. A. Kruper, and N. Frenkel. 1988. Herpes simplex virus virion host shutoff function. J Virol 62:912-21.
34. Leone, V., A. di Palma, P. Ricchi, F. Acquaviva, M. Giannouli, A. M. Di Prisco, F. Iuliano, and A. M. Acquaviva. 2007. PGE2 inhibits apoptosis in human adenocarcinoma Caco-2 cell line through Ras-PI3K association and cAMP-dependent kinase A activation. Am J Physiol Gastrointest Liver Physiol 293:G673-81.
35. Li, M., J. Xie, L. Cheng, B. Chang, Y. Wang, X. Lan, D. Zhang, Y. Yin, and N. Cheng. 2008. Suppression of invasive properties of colorectal carcinoma SW480 cells by 15-hydroxyprostaglandin dehydrogenase gene. Cancer Invest 26:905-12.
36. Liu, B., L. Qu, and H. Tao. Cyclo-oxygenase 2 up-regulates the effect of multidrug resistance. Cell Biol Int 34:21-5.
37. Manservigi, R., R. Argnani, and P. Marconi. HSV Recombinant Vectors for Gene Therapy. Open Virol J 4:123-56.
38. Marrogi, A. J., W. D. Travis, J. A. Welsh, M. A. Khan, H. Rahim, H. Tazelaar, P. Pairolero, V. Trastek, J. Jett, N. E. Caporaso, L. A. Liotta, and C. C. Harris. 2000. Nitric oxide synthase, cyclooxygenase 2, and vascular endothelial growth factor in the angiogenesis of non-small cell lung carcinoma. Clin Cancer Res 6:4739-44.
39. Matzinger, P. 1998. An innate sense of danger. Semin Immunol 10:399-415.
40. Matzinger, P. 2002. An innate sense of danger. Ann N Y Acad Sci 961:341-2.
41. Matzinger, P. 1994. Tolerance, danger, and the extended family Annu Rev Immunol 12:991-1045.
42. Myung, S. J., R. M. Rerko, M. Yan, P. Platzer, K. Guda, A. Dotson, E. Lawrence, A. J. Dannenberg, A. K. Lovgren, G. Luo, T. P. Pretlow, R. A. Newman, J. Willis, D. Dawson, and S. D. Markowitz. 2006. 15-Hydroxyprostaglandin dehydrogenase is an in vivo suppressor of colon tumorigenesis. Proc Natl Acad Sci USA 103:12098-102.
43. Nakamori, M., X. Fu, R. Rousseau, S. Y. Chen, and X. Zhang. 2004. Destruction of nonimmunogenic mammary tumor cells by a fusogenic oncolytic herpes simplex virus induces potent antitumor immunity. Mol Ther 9:658-65.
44. Ochoa, A. C., A. H. Zea, C. Hernandez, and P. C. Rodriguez. 2007. Arginase, prostaglandins, and myeloid-derived suppressor cells in renal cell carcinoma. Clin Cancer Res 13:721s-726s.
45. Oroskar, A. A., and G. S. Read. 1989. Control of mRNA stability by the virion host shutoff function of herpes simplex virus. J Virol 63:1897-906.
46. Ostrand-Rosenberg, S. 2008. Immune surveillance: a balance between protumor and antitumor immunity. Curr Opin Genet Dev 18:11-8.
47. Ostrand-Rosenberg, S. Myeloid-derived suppressor cells: more mechanisms for inhibiting antitumor immunity. Cancer Immunol Immunother 59:1593-600.
48. Pasieka, T. J., B. Lu, S. D. Crosby, K. M. Wylie, L. A. Morrison, D. E. Alexander, V. D. Menachery, and D. A. Leib. 2008. Herpes simplex virus virion host shutoff attenuates establishment of the antiviral state. J Virol 82:5527-35.
49. Prestwich, R. J., F. Errington, R. M. Diaz, H. S. Pandha, K. J. Harrington, A. A. Melcher, and R. G. Vile. 2009. The case of oncolytic viruses versus the immune system: waiting on the judgment of Solomon. Hum Gene Ther 20:1119-32.
50. Pulaski, B. A., and S. Ostrand-Rosenberg. 2001. Mouse 4T1 breast tumor model. Curr Protoc Immunol Chapter 20:Unit 20 2.
51. Read, G. S., and N. Frenkel. 1983. Herpes simplex virus mutants defective in the virion-associated shutoff of host polypeptide synthesis and exhibiting abnormal synthesis of alpha (immediate early) viral polypeptides. J Virol 46:498-512.
52. Read, G. S., B. M. Karr, and K. Knight. 1993. Isolation of a herpes simplex virus type 1 mutant with a deletion in the virion host shutoff gene and identification of multiple forms of the vhs (UL41) polypeptide. J Virol 67:7149-60.
53. Rodriguez, P. C., C. P. Hernandez, D. Quiceno, S. M. Dubinett, J. Zabaleta, J. B. Ochoa, J. Gilbert, and A. C. Ochoa. 2005. Arginase I in myeloid suppressor cells is induced by COX-2 in lung carcinoma. J Exp Med 202:931-9.
54. Samady, L., E. Costigliola, L. MacCormac, Y. McGrath, S. Cleverley, C. E. Lilley, J. Smith, D. S. Latchman, B. Chain, and R. S. Coffin. 2003. Deletion of the virion host shutoff protein (vhs) from herpes simplex virus (HSV) relieves the viral block to dendritic cell activation: potential of vhs-HSV vectors for dendritic cell-mediated immunotherapy. J Virol 77:3768-76.
55. Santos, F. C., S. A. de Oliveira, S. Setubal, L. A. Camacho, T. Fullace, J. P. Leite, and L. G. Velarde. 2006. Seroepidemiological study of herpes simplex virus type 2 in patients with the acquired immunodeficiency syndrome in the city of Niteroi, R10 de Janeiro, Brazil. Mem Inst Oswaldo Cruz 101:315-9.
56. Schek, N., and S. L. Bachenheimer. 1985. Degradation of cellular mRNAs induced by a virion-associated factor during herpes simplex virus infection of Vero cells. J Virol 55:601-10.
57. Sharma, S., S.C. Yang, L. Zhu, K. Reckamp, B. Gardner, F. Baratelli, M. Huang, R. K. Batra, and S. M. Dubinett. 2005. Tumor cyclooxygenase-2/prostaglandin E2-dependent promotion of FOXP3 expression and CD4+CD25+T regulatory cell activities in lung cancer. Cancer Res 65:5211-20.
58. Sinha, P., V. K. Clements, A. M. Fulton, and S. Ostrand-Rosenberg. 2007. Prostaglandin E2 promotes tumor progression by inducing myeloid-derived suppressor cells. Cancer Res 67:4507-13.
59. Smiley, J. R. 2004. Herpes simplex virus virion host shutoff protein: immune evasion mediated by a viral RNase? J Virol 78:1063-8.
60. Smith, T. J., L. A. Morrison, and D. A. Leib. 2002. Pathogenesis of herpes simplex virus type 2 virion host shutoff (vhs) mutants. J Virol 76:2054-61.
61. Sorenson, C. M., P. A. Hart, and J. Ross. 1991. Analysis of herpes simplex virus-induced mRNA destabilizing activity using an in vitro mRNA decay system. Nucleic Acids Res 19:4459-65.
62. Strelow, L., T. Smith, and D. Leib. 1997. The virion host shutoff function of herpes simplex virus type 1 plays a role in corneal invasion and functions independently of the cell cycle. Virology 231:28-34.
63. Strelow, L. I., and D. A. Leib. 1996. Analysis of conserved domains of UL41 of herpes simplex virus type 1 in virion host shutoff and pathogenesis. J Virol 70:5665-7.
64. Strelow, L. I., and D. A. Leib. 1995. Role of the virion host shutoff (vhs) of herpes simplex virus type 1 in latency and pathogenesis. J Virol 69:6779-86.
65. Strom, T., and N. Frenkel. 1987. Effects of herpes simplex virus on mRNA stability. Virol 61:2198-207.

66. Tai, H. H., M. Tong, and Y. Ding. 2007. 15-hydroxyprostaglandin dehydrogenase (15-PGDH) and lung cancer. Prostaglandins Other Lipid Mediat 83:203-8.
67. Tatsuwaki, H., T. Tanigawa, T. Watanabe, H. Machida, H. Okazaki, H. Yamagami, M. Shiba, K. Watanabe, K. Tominaga, Y. Fujiwara, N. Oshitani, K. Muguruma, T. Sawada, K. Hirakawa, K. Higuchi, and T. Arakawa. 2009. Reduction of 15-hydroxyprostaglandin dehydrogenase expression is an independent predictor of poor survival associated with enhanced cell proliferation in gastric adenocarcinoma. Cancer Sci.
68. Tischer, B. K., J. von Einem, B. Kaufer, and N. Osterrieder. 2006. Two-step red-mediated recombination for versatile high-efficiency markerless DNA manipulation in Escherichia coli. Biotechniques 40:191-7.
69. Trgovcich, J., D. Johnson, and B. Roizman. 2002. Cell surface major histocompatibility complex class II proteins are regulated by the products of the gamma(1)34.5 and U(L)41 genes of herpes simplex virus 1. J Virol 76:6974-86.
70. Tseng-Rogenski, S., J. Gee, K. W. Ignatoski, L. P. Kunju, A. Bucheit, H. J. Kintner, D. Morris, C. Tallman, J. Evron, C. G. Wood, H. B. Grossman, C. T. Lee, and M. Liebert. Loss of 15-Hydroxyprostaglandin Dehydrogenase Expression Contributes to Bladder Cancer Progression. Am J. Pathol.
71. Tsujii, M., S. Kawano, S. Tsuji, H. Sawaoka, M. Hori, and R. N. DuBois. 1998. Cyclooxygenase regulates angiogenesis induced by colon cancer cells. Cell 93:705-16.
72. Wacholtz, M. C., R. Minakuchi, and P. E. Lipsky. 1991. Characterization of the 3′,5′-cyclic adenosine monophosphate-mediated regulation of IL2 production by T cells and Jurkat cells. Cell Immunol 135:285-98.
73. Wanebo, H. J., R. Pace, S. Hargett, D. Katz, and J. Sando. 1986. Production of and response to interleukin-2 in peripheral blood lymphocytes of cancer patients. Cancer 57:656-62.
74. Yan, M., R. M. Rerko, P. Platzer, D. Dawson, J. Willis, M. Tong, E. Lawrence, J. Lutterbaugh, S. Lu, J. K. Willson, G. Luo, J. Hensold, H. H. Tai, K. Wilson, and S. D. Markowitz. 2004. 15-Hydroxyprostaglandin dehydrogenase, a COX-2 oncogene antagonist, is a TGF-beta-induced suppressor of human gastrointestinal cancers. Proc Natl Acad Sci U S A 101:17468-73.
75. Zelus, B. D., R. S. Stewart, and J. Ross. 1996. The virion host shutoff protein of herpes simplex virus type 1: messenger ribonucleolytic activity in vitro. J Virol 70:2411-9.
76. Zou, W. 2005. Immunosuppressive networks in the tumour environment and their therapeutic relevance. Nat Rev Cancer 5:263-74.
77. Advani, S. J., S. Chung, S. Yan, G. Y. Gillespie, J. Markert, R. J. Whitley, B. Roizman, R. R. Weichselbaum. 1999. Replication-competent, nonueuroinvasive genetically engineered herpes virus is highly effective in the treatment of therapy-resistant experimental human tumors. Cancer Res 59:2055-2058.
78. Bryant, K. F., E. R. Macari, N. Malik, M. Boyce, J. Yuan, and D. M. Coen. 2008. ICP34.5-Dependent and-Independent acitivites of salubrinal in herpes simplex virus-1 infected cells. Virology 30: 197-204.
79. Foster, T. P., J. M. Melancon, J. D. Baines, and K. G. Kousoulas. 2004. The herpes simplex virus type 1 UL20 protein modulates membrane fusion events during cytoplasmic viron morphogenesis and viral-induced cell fusion. J. Virology 78: 5347-5357.
80. Foster, T. P., V. N. Chouljenko, and K. G. Kousoulas. 2008. Functional and physical interactions of the herpes simplex virus type 1 UL20 membrane protein with glycoprotein K. J. Virology 82: 6310-6323.
81. Jing, X., M. Cerveny, K. Yang, and B. He. 2004. Replication of herpes simplex virus 1 depends on the γ₁34.5 functions that facilitate virus response to interferon and egress in the different stages of productive interferon. J. Virology 78: 7653-7666.
82. Papadakis, E. D., S. A. Nicklin, A. H. Baker, and S. J. White. 2004. Promoters and control elements: designing expression cassettes for gene therapy. Current Gene Therapy 4: 89-113.
83. Samoto, K., M. Ehtesham, G-C Perng, K. Hashizume, S. L. Wechsler, A. B. Nesburn, K. L. Black, and J. S. Yu. 2002. A herpes simplex virus type 1 mutant with γ34.5 and LAT deletions effectively oncolyses human U87 glioblastomas in nude mice. Neurosurgery 50: 599-606.
84. Wong, R. J., M.-K. Chan, Z. Yu, R. A. Ghossein, I. Ngai, P. S. Adusumilli, B. M. Stiles, J. P. Shah, B. Singh, and Y. Fong. 2004. Angiogenesis inhibition by an oncolytic herpes virus expressing interleukin 12. Clinical Cancer Research 10: 4509-4516.
85. Yuhasz, S, and J. G. Stevens. 1993. Glycoprotein B is a specific determinant of herpes simplex virus type 1 neuroinvasiveness. J. Virology 67: 5948-5954.
86. Nagano, S. et al. 2008. Cancer cell death enhances the penetration and efficacy of oncolytic herpes simplex virus in tumors. Cancer Res. 68: 3795-3802.
87. Passer, B. J. et al. 2009. Oncolytic herpes simplex virus vectors and taxanes synergize to promote killing of prostate cancer cells. Cancer Gene Ther. 16: 551-560.
88. Lin, S. F. et al. 2008. Synergy of a herpes oncolytic virus and paclitaxel for anaplastic thyroid cancer. Clin Cancer Res. 14: 1519-1528.

The complete disclosures of all references cited in this specification are hereby incorporated by reference. Also incorporated by reference are the complete disclosures of the following: (1) J. D. Walker et al., “Oncolytic and immunomudulatory therapy with a novel herpes simplex-1-based vector armed with ability to inhibit prostaglandin synthesis,” an Abstract and Poster presented at the LCRC Annual Scientific Retreat 2010, March 26^{th} and 27^{th}, New Orleans, La.; and (2) J. D. Walker et al., “Oncolytic herpes simplex virus type-1 encoding 15-prostaglandin dehydrogenase mitigates immune suppression and reduces ectopic primary and metastatic breast cancer in mice,” a manuscript submitted to the Journal of Virology, January 2011.

1. A recombinant, replicating herpes simplex virus whose chromosome concurrently lacks the coding sequence for a single γ₁ 34.5 gene and for Viral Host Shutoff gene and comprises a mutated coding sequence in the UL27 (gB) gene, wherein presence of said mutated coded sequence increases the ability of the virus to promote virus-induced cell fusion.

2. A recombinant, replicating herpes simplex virus as in claim 1, additionally comprising one or more exogenous genes.

3. The virus as in claim 2, wherein the one or more exogenous genes code for one or more proteins selected from the group consisting of a protein that decreases the viral-caused immunosuppression, a protein that presents an cancer or vial antigen to the host, a protein that stimulates the host immune response, and a protein that decreases angiogenesis.

4. The virus of claim 2, wherein one or more exogenous genes code for one or more proteins selected from the group consisting of prostaglandin dehydrogenase, interleukin-2 (IL-2), interleukin-4 (IL-4), interleukin-10 (IL-10), interleukin-12 (IL-12), granulocytes-macrophage colony stimulator factor (GM-CSF), Interferon-γ (INF-γ), tumor necrosis factor alpha (TNFα), co-stimulatory surface antigen (CD80), endostatin, trichostatin A (TSA), vasculostatin (Vstat120), brain-specific angiogenesis inhibitor 1 (BAI1), HER2/neu (ErbB-2) antigen, carcinoembryonic antigen (CEA1), MUC-1, epithelial tumor antigen (ETA), Epstein Barr viral antigens, Papilloma viral antigens, additional herpes simplex viral antigens, and other known viral antigens.

5. The virus of claim 2, wherein the exogenous gene additionally comprises one or more exogenous promoter genes that controls expression of the one or more exogenous genes.

6. The virus of claim 5, wherein the one or more promoter genes are active only in a cancer cell.

7. The virus of claim 6, wherein the one or more promoter genes are selected from the group consisting of cytomegalovirus immediate early promoter (CMV-IE), simian virus 40 promoter (SV40), the Rous sarcoma virus long terminal repeat promoter (RSV-LTR), Moloney murine leukemia virus (MoMLV) LTR, other retroviral LTR promoters, phosphoglycerate kinase (PGK) promoter, Elongation factor 1a (ER1a) promoter, Her-2/neu (erbB2) promoter, carcinoembryonic antigen (CEA) promoter, PSA promoter, and probasin (ARR2PB) promoter).

8. The virus as in claim 1, wherein the virus is herpes simplex virus type 1.

9. (canceled)

10. (canceled)

11. An oncolytic, recombinant, replicating herpes virus that comprises a mammalian prostaglandin dehydrogenase gene.

12. The oncolytic virus of claim 11, whose chromosome concurrently lacks the coding sequence for a Viral Host Shutoff gene.

13. A method for treating a tumor in a mammal, comprising administering to the mammal with the tumor a therapeutic amount of the herpes simplex virus as in claim 1.

14. The method of claim 13, additionally comprising administering to the mammal one or more therapies selected from the group consisting of radiation therapy and chemotherapy.

15. The method of claim 13, additionally comprising administering to the mammal one or more drugs selected from the group consisting of a known cancer drug and a non-steroidal anti-inflammatory drug.

16. The method of claim 13, additionally comprising administering to the mammal one or more drugs selected from the group consisting of docetaxel, paclitaxel, fludarabine, CD20 antibody, histone deacetylase inhibitor, doxorubicin, cisplatin, Trichostatin A, bevacizumab, aspirin, ibuprofen, celecoxib, rofecoxib, salsalate, sodium salicylate,

17. The method of claim 13, additionally comprising administering paxlitaxel to the mammal.

18. The method of claim 13, additionally comprising administering ibuprofen to the mammal.

19. The virus as in claim 1, wherein the mutated coded sequence in the UL27(gB) gene is gBsyn3.

20. The virus of claim 2, wherein the one or more exogenous genes code for prostaglandin dehydrogenase.

21. The virus of claim 4, wherein the one or more exogenous genes code for additional herpes simplex viral antigens or other known viral antigens.

22. A method for treating a tumor in a mammal, comprising administering to the mammal with the tumor a therapeutic amount of the virus as in claim 11.

23. A method to enhance an immune response against a herpes simplex virus, said method comprising administering to the mammal with the tumor a therapeutic amount of the virus as in claim 1.

24. A viral vector comprising the virus as in claim 1.

25. A vaccine comprising the virus as in claim 21.


SEQUENCE LISTING (5 sequences)

SEQ ID NO: 1 - DNA, 21 bp, Artificial sequence
  Synthetic primer
aggagtgttcgagtcgtgtct

SEQ ID NO: 2 - DNA, 20 bp, Artificial sequence
  Synthetic primer
tgggactatatgagcccgag

SEQ ID NO: 3 - DNA, 21 bp, Artificial sequence
  Synthetic primer
cctgctgaaggagagtatgcg

SEQ ID NO: 4 - DNA, 22 bp, Artificial sequence
  Synthetic primer
atgctcgccgtccgttccctgc

SEQ ID NO: 5 - DNA, 22 bp, Artificial sequence
  Synthetic primer
atcaaacaggcgcctctggatc